(19) United States
(12) Patent Application Publication
(10) Pub. No.: US 2026/0166110 A1
(43) Pub. Date: Jun. 18, 2026

(54) Noni (Morinda citrifolia) Concentrate as an Anti-diabetic Nutraceutical

(71) Applicant: Northern Marianas College, Saipan, MP (US)

(72) Inventors: Md Zaidul Islam Sarker, Saipan, MP (US); Md Abdur Rashid Mia, Saipan, MP (US); Patricia Coleman, Saipan, MP (US)

(73) Assignee: Northern Marianas College, Saipan, MP (US)

(21) Appl. No.: 19/464,600

(22) Filed: Jan. 30, 2026

Related U.S. Application Data
(63) Continuation of application No. 19/286,182, filed on Jul. 30, 2025.

(51) Int. Cl. A61K 36/746; A61K 31/133; A61K 31/192; A61K 31/231; A61K 31/232; A61K 31/352; A61K 31/5375; A61K 31/56; A61K 31/7048; A61P 3/10; B01D 11/02

(52) U.S. Cl. CPC A61K 36/746; A61K 31/133; A61K 31/192; A61K 31/231; A61K 31/232; A61K 31/352; A61K 31/5375; A61K 31/56; A61K 31/7048; A61P 3/10; B01D 11/0203; B01D 11/0288; A61K 2236/37; A61K 2236/51; A61K 2236/53

(57) ABSTRACT

A composition of noni (Morinda citrifolia) concentrate as an anti-diabetic nutraceutical prepared using supercritical carbon dioxide extraction. The anti-diabetic nutraceutical is in a form of supercritical carbon dioxide extract and has a strong α-glucosidase inhibition activity characterized by IC₅₀ lower than 50 μg/mL measured in vitro using isolated α-glucosidase enzyme. The extract is useful as anti-diabetic remedy with no acute or sub-acute toxicity and it also lowers blood total cholesterol, LDL-C and triglycerides and increases HDL-C in diabetic mammals.

### CROSS-REFERENCE TO RELATED APPLICATIONS

This application is a continuation of U.S. application Ser. No. 19/286,182, filed Jul. 30, 2025, the disclosure of which is incorporated by reference herein in its entirety.

### FEDERALLY SPONSORED RESEARCH AND DEVELOPMENT

This invention was made with government support under grant no. NI23HFPXXXXXG057 awarded by the U.S. Department of Agriculture's National Institute of Food and Agriculture. The government has certain rights in the invention.

### BACKGROUND OF THE INVENTION

Supercritical fluid extraction (SFE), particularly supercritical carbon dioxide (scCO₂) extraction, has recently gained popularity as a green method for the isolation and identification of bioactive compounds from natural sources. scCO₂ is an efficient and selective extraction method that can be fine-tuned rather easily regarding temperature, pressure, and co-solvents to extract the desired product. When applying the technique, scCO₂ extraction employs carbon dioxide in the supercritical state, and other extraction solvents, which enables accurate control of the extraction conditions and reduces the content of the solvent to a minimum in the final product. It is commonly used to extract phenolics, flavonoids, alkaloids, steroids, anthraquinones, and fatty acids from different plants under different conditions. However, the use of this approach in the research of Morinda citrifolia (also known as noni) has not been comprehensively investigated.

Morinda citrifolia is a tropical plant known for its therapeutic qualities and extensive use in traditional medicine. Studies have identified several bioactive compounds in the plant such as phenolics, flavonoids, anthraquinones and scopoletin, which possess antioxidant, anti-inflammatory, anticancer and anti-diabetic effects. Traditional techniques for isolating these compounds often use organic solvents, which have environmental and health hazards. Furthermore, these techniques exhibit less efficacy in safeguarding critical compounds, resulting in decreased bioactivity within the extracts. To be medically viable, the extract should show the health benefits at reasonable doses that can be given to the patient. Many conventional extraction methods do not yield strong extracts to be viable as a medication or dietary supplement.

Antioxidants play a vital role in combating oxidative stress, which is linked to numerous diseases including diabetes. The extracts of Morinda citrifolia has been found to possess high antioxidant properties mainly due to the high phenolic content. The plant's effectiveness in treating diabetes is linked to its ability to inhibit the activity of the α-glucosidase (alpha-glucosidase) enzyme and regulate the activity of PPAR-γ, thus improving glucose handling. However, most of these studies rely on extracts prepared using conventional methods and there is little information concerning the bioactivity of extracts obtained by scCO₂ extraction. Simamora et al (2019) characterized the fermented juice microbiologically and chemically evaluated its α-glucosidase inhibition and radical scavenging activities in vitro. The fruit of Morinda citrifolia was fermented and the fruit juice was obtained and evaluated for its radical scavenging activity and in vitro anti-diabetic activity on α-glucosidase. The fermented fruit juice showed good α-glucosidase inhibitory and antioxidant activities with IC₅₀ (half-maximal inhibitory concentration) of 28.99 and 14.09 μg GAE/mL (Gallic Acid equivalent), respectively. The kinetic study showed a non-competitive inhibition on α-glucosidase. The combination of the juice with Acarbose at higher concentrations produced an additive effect on α-glucosidase, but at lower concentrations, an antagonistic effect was observed.

Diabetes mellitus is a metabolic disorder of glucose metabolism. The management of blood glucose level is the hallmark in the treatment of this disease. This may be achieved through the use of oral hypoglycemic drugs such as biguanides, insulin secretagogues, and α-glucosidase inhibitors. There is limited information about the extraction of α-glucosidase inhibitors from noni plant. Dewi et al (2022) extracted α-glucosidase inhibitors from the ethyl acetate extract of fermented noni fruit juice. Separation and purification yielded two compounds identified as scopoletin and quercetin with IC₅₀ values of respectively 61.93 and 21.65 μM for α-glucosidase, 844.06 and 35.23 μM for DPPH (2,2-diphenyl-1-picrylhydrazyl) free radical scavenger effect. This study suggested that scopoletin and quercetin can be used as quality control biomarkers for products produced from fermented Morinda citrifolia juice extract.

Khamis et al (2015) used the ethanolic extract of the noni fruit to study the in vitro α-glucosidase inhibition. They found that the extract at 1 mg/ml concentration had mild inhibitory activity on α-glucosidase with percentage of inhibition at 57% which was comparable to that of the standard Acarbose at concentration of 1.0 mg/ml (Percentage of inhibition of 57.7%).

Phanumartwiwath et al (2022) evaluated the inhibition of α-glucosidase from the root of noni extracted with increasing polarity of solvents (hexane, dichloromethane, ethyl acetate and ethanol). Among the four crude extracts, the dichloromethane root extract had the highest α-glucosidase inhibitory activity against rat intestinal sucrase with an IC₅₀ value of 0.943 mg/ml. Furthermore, this extract inhibited yeast α-glucosidase with an IC₅₀ value of 0.646 mg/ml, having greater activity than the standard drug, Acarbose (IC_{50=1.122} mg/ml).

The various geographic and climatic conditions prevailing in the CNMI (Commonwealth of the Northern Mariana Islands) result in different phytochemicals being present in Morinda citrifolia grown locally. Despite such differences, there is no research that focuses on determining the optimum scCO₂ extraction conditions for Morinda citrifolia grown in the CNMI and comparing the anti-diabetic and antioxidant potential of the extracts with those obtained using conventional techniques. There is also lack of knowledge related to the extraction of α-glucosidase inhibitors from noni using scCO₂. To this end, a new strategy is still needed to be developed to enable scCO₂ extraction of bioactive compounds with enhanced antioxidant and anti-diabetic potential from Morinda citrifolia, especially the one grown in CNMI.

### SUMMARY OF THE INVENTION

This invention provides a method for extracting active ingredients from Morinda citrifolia (noni) using a modified scCO₂ method with more focus on the anti-diabetic ingredients. More specifically, this invention provides a method to prepare an extract with a strong α-glucosidase inhibitor extract from Morinda citrifolia (noni) fruits, leaves and seeds using scCO₂. Compared to the conventional extraction methods like Soxhlet method, our scCO₂ method resulted in more potent extracts in terms of antioxidant and anti-diabetic activities from different parts of the plant including fruits, leaves, and seeds; therefore, we refer it as Noni Essence or Noni Concentrate.

The extraction was performed on the ground Morinda citrifolia samples using supercritical carbon dioxide (scCO₂) under a pressure from 15 to 40 MPa, co-solvent (ethanol) 10 to 30% and a temperature from 30° C. to 80° C. The optimized parameters of scCO₂ were 27.5 MPa, temperature 55° C. and 20% co-solvent (ethanol) to obtain extracts with strong bioactivity effects compared to the conventional method of Soxhlet extraction. The optimized scCO₂ extracts exhibited higher antioxidant and α-glucosidase inhibitory activities, were found non-toxic and exhibited anti-diabetic effects in animal models.

### BRIEF DESCRIPTION OF DRAWINGS

FIG. 1: Microscopic image (40× magnification) showing the effect of noni fruit extracts on mice liver, heart, kidney, lungs, and brain in the acute toxicity study. G1=normal control (only water), G2=1875 mg/kg bw (body weight), G3=3750 mg/kg bw, G4=7500 mg/kg bw.

FIG. 2: Microscopic image (40× magnification) showing the effect of noni leaves extracts on mice liver, heart, kidney, lungs, and brain in the acute toxicity study. G1=Normal control (only water), G2=1250 mg/kg bw, G3=2500 mg/kg bw, G4=5000 mg/kg bw.

FIG. 3: Microscopic image (40× magnification) showing the effect of noni seed extracts on mice liver, heart, kidney, lungs, and brain in the acute toxicity study. G1=Normal control (only water), G2=5 ml/kg bw, G3=10 ml/kg bw, G4=20 ml/kg bw.

FIG. 4: Microscopic image (40× magnification) showing the effect of noni fruit extracts on mice liver, heart, kidney, lungs, and brain in the sub-acute toxicity study. G1=Normal control (only water), G2=938 mg/kg bw, G3=1875 mg/kg bw, G4=3750 mg/kg bw.

FIG. 5: Microscopic image (40× magnification) showing the effect of noni leaves extracts on mice liver, heart, kidney, lungs, and brain in the sub-acute toxicity study. G1=Normal control (only water), G2=625 mg/kg bw, G3=1250 mg/kg bw, G4=2500 mg/kg bw.

FIG. 6: Microscopic image (40× magnification) showing the effect of noni seed extracts on mice liver, heart, kidney, lungs, and brain in the sub-acute toxicity study. G1=Normal control (only water), G2=2.5 ml/kg bw, G3=5 ml/kg bw, G4=10 mL/kg bw.

FIG. 7: Effect of Noni fruit extracts on fasting blood glucose. G1-Normal mice, G2-Noni fruit treated (200 mg/kg bw) on normal mice, G3-Diabetic control group (streptozotocin (STZ) treated), G4-Drug control (Glibenclamide 5 mg/kg bw), G5-Noni fruit treated (200 mg/kg bw) on diabetic mice, G6-Noni fruit treated (400 mg/kg bw) on diabetic mice.

FIG. 8: Effect of Noni leaves extracts on fasting blood glucose. G1-Normal mice, G2-Noni leaves treated (200 mg/kg bw) on normal mice, G3-Diabetic control group (STZ treated), G4-Drug control (Glibenclamide 5 mg/kg bw), G5-Noni leaves treated (200 mg/kg bw) on diabetic mice, G6-Noni leaves treated (400 mg/kg bw) on diabetic mice.

FIG. 9: Microscopic image (40× magnification) showing the effect of noni fruit extracts on mice pancreas, liver, heart and kidney in the anti-diabetic study. G1-Normal mice, G2-Noni fruit treated (200 mg/kg bw) on normal mice, G3-Diabetic control group (STZ treated), G4-Drug control (Glibenclamide 5 mg/kg bw), G5-Noni fruit treated (200 mg/kg bw) on diabetic mice, G6-Noni fruit treated (400 mg/kg bw) on diabetic mice.

FIG. 10: Microscopic image (40× magnification) showing the effect of noni leaves extracts on mice pancreas, liver, heart and kidney in the anti-diabetic study. G1-Normal mice, G2-Noni leaves treated (200 mg/kg bw) on normal mice, G3-Diabetic control group (STZ treated), G4-Drug control (Glibenclamide 5 mg/kg bw), G5-Noni leaves treated (200 mg/kg bw) on diabetic mice, G6-Noni leaves treated (400 mg/kg bw) on diabetic mice.

### DETAILED DESCRIPTION OF THE INVENTION

The present invention provides a method for preparing scCO₂ extracts from noni having anti-diabetic effects with a strong α-glucosidase inhibition activity. The scCO₂ extracts were prepared from noni fruits, leaves, and seeds. These extracts were safe to use where they exhibited no toxicity in animal models even at high doses. Other than the anti-diabetic effects, the scCO₂ extracts exhibited strong anti-oxidant activities. Our extraction method was able to extract several compounds with anti-diabetic effects and they were identified for the first time from Noni.

The following example describes in details the preparation methods and the tests performed on the extracts. Some variations to the parameters and materials can be performed by a skilled person in the art without undue experimentation.

### Example A: Noni Scco₂ Extracts' Preparation and Testing in Comparison with Conventional Soxhlet Extraction

### 1. Materials and Methods

### 1.1 Raw Materials Collection

Morinda citrifolia leaves, fruits, and seeds were collected from the Commonwealth of the Northern Mariana Islands (CNMI). After collecting, the Morinda citrifolia leaves were dried under sunlight and crushed with a Professional Nutrition Blender (model: 10t2.) to make it in powder form. In addition, Morinda citrifolia mature fruits flesh was separated from seeds. Later, fruits flesh and seeds were separately dried and ground to make a fine powder.

### 1.2 Extraction Methods

### 1.2.1 Soxhlet Extraction

The conditions of the Soxhlet extraction process were the following—
Solvent: Ethanol (food grade, 95% purity).
Solvent volume: 200 mL.
Sample volume: 20 g.
Sample type: dry powder.
Extraction time: 8 hours.
Temperature: 85° C.
Drying method: Rotary evaporator and oven. The filtrate obtained after Soxhlet extraction was concentrated to evaporate ethanol by using a rotary evaporator (Buchi, B-490, Postfach, Flawil, Switzerland) at 40° C. under reduced pressure. The evaporated extract was then dried in an oven at 40° C. for 24 hr to complete drying.

The yield was recorded by the following equation (eq. 1)

$$Yield ( \% ) = ( Mass of extract ( g ) after evaporate ) / ( Mass of dry sample ( g ) ) \times 100 \qquad eq . 1$$

1.2.2 Supercritical Carbon Dioxide (scCO₂) Extraction

The scCO₂ extraction process was conducted using a supercritical CO₂ extractor (Model: SFE-1000) on a series of preliminary trials whereby the co-solvent, pressure, and temperature were selected at various ranges. Design expert 7.0 software was used to select the number of runs based on the variables (pressure: 15 to 40 MPa, co-solvent (ethanol): 10 to 30% of the plant material weight, temperature: 30° C. to 80° C.) shown in Table 1. At first, the leaves sample was run to select the best condition for obtaining the optimum yield. Later, we ran the fruits and seeds based on the optimum conditions. Based on the optimized condition, fruits and seeds were run at optimized conditions: 55° C. temperature, 27.5 MPa pressure and 20% co-solvent. After completing the evaporation of the solvent, the dried extracts were calculated for the percentage of yields and finally stored at −20° C. in the refrigerator for further analysis.

scCO₂ Extraction Conditions:
Gas: CO₂.
Extraction time: 6 hr.
Flowrate: 0.7-0.9 kg/hr.
Solvent: Ethanol (food grade, 95% purity).
Drying method: Rotary evaporator and oven. The filtrate obtained after scCO₂ extraction was concentrated to evaporate solvent by using a rotary evaporator (Buchi, B-490, Postfach, Flawil, Switzerland) at 40° C. under reduced pressure. The evaporated extract was then dried in an oven at 40° C. for 24 hr to complete drying. The fruit extract was semi-solid and the leaves extract was in powder form, while the extract from the seeds was in liquid form.

TABLE 1
The selected temperature, pressure, and solvent
derived from design expert software.

| | Temperature | Pressure | Co-solvent |
|---|---|---|---|
| Run No. | (° C.) | (MPa) | (%) |
| 1 | 55 | 40 | 10 |
| 2 | 30 | 27.5 | 30 |
| 3 | 55 | 27.5 | 20 |
| 4 | 80 | 27.5 | 30 |
| 5 | 55 | 27.5 | 20 |
| 6 | 80 | 15 | 20 |
| 7 | 55 | 15 | 30 |
| 8 | 55 | 27.5 | 20 |
| 9 | 55 | 15 | 10 |
| 10 | 30 | 27.5 | 10 |
| 11 | 30 | 40 | 20 |
| 12 | 55 | 27.5 | 20 |
| 13 | 30 | 15 | 20 |
| 14 | 55 | 27.5 | 20 |
| 15 | 80 | 27.5 | 10 |
| 16 | 55 | 40 | 30 |
| 17 | 80 | 40 | 20 |

### 1.3 Bioactivity Analysis

### 1.3.1 Total Phenolic Content

Total phenolic content (TPC) of Morinda citrifolia leaves, fruits, and seeds extracts was analyzed in order to obtain a standard curve for gallic acid. The total phenolics were expressed as mg gallic acid equivalent (GAE) per g of dry powder sample.

### 1.3.2 Total Flavonoid Content

A colorimetric procedure was employed to assess the total flavonoids in noni leaves, fruits, and seeds extracts, as described by established methods in the literature. Quercetin was used to obtain a calibration curve following the same procedures as the extract. The quantities of total flavonoids were expressed as mg quercetin equivalents per gram (mg QE/g) of the dry extract.

### 1.3.3 DPPH Free Radical Scavenging Activity

A commonly used 1,1-diphenyl-2-picrylhydrazyl (DPPH) method was used to assess radical scavenging properties of Morinda citrifolia leaves, fruits and seeds extracts. Absorbance was measured at 517 nm and IC₅₀ was calculated based on the % of inhibition, which is followed by the following equation (eq. 2):

$$DPPH Scavenging activity ( \% ) = ( ( A 0 - A 1 ) / A 0 ) \times 100 \qquad eq . 2$$

where, A0 represents the blank absorbance (negative control) and A¹ is the absorbance of the samples.

### 1.3.4 Ferric Reducing Antioxidant Power (FRAP)

The FRAP assay was estimated based on established methods in the literature with some modifications. The absorbance of each sample was determined using a microplate reader at 593 nm. A calibration curve was formed using Ascorbic Acid and FRAP activity was expressed as Ascorbic Acid equivalent (AAE)/g of dried sample.

### 1.3.5 α-Glucosidase Inhibitory Activity

The α-glucosidase inhibitory activity was determined of the extracts according to the established method. The different concentrations of extracts and positive control were prepared in dimethyl sulfoxide (DMSO). About 10 μL of freshly prepared stock solutions were added with 30 mM of phosphate buffer. The enzyme solution was prepared by mixing 1 mg of the enzyme with 13.9 mL of 50 mM phosphate buffer (pH 6.5). The substrate of p-nitrophenyl-α-D-glucopyranose was dissolved in 50 mM of the previously prepared buffer. After that, the plate was placed for 15 min at laboratory room temperature. A positive, negative control, blank sample, and blank positive control were prepared accordingly. Glycine was used to stop the reaction. The absorbance was read at 405 nm using a microplate reader. The IC₅₀ was analyzed using linear regression analysis and the inhibitory activity (%) of each extract was calculated using the following equation (eq. 3):

$$Inhibition ( \% ) = ( ( A 0 - A 1 ) / A 0 ) \times 100 \qquad eq . 3$$

where, A0 is the absorbance of the negative control, and A¹ is the absorbance of the sample or positive control.

### 1.4 Identification of Bioactive Compounds by GC-MS Analysis

All extracts of Morinda citrifolia leaves, fruits, and seeds were identified by gas chromatography-mass spectroscopy (GC-MS) using the following method: Agilent 6890 N2 gas chromatography coupled with a mass selective detector MS-5973 (model: 7250, Agilent Technologies, USA) was used. About 1 μl of the experimental sample was injected into a SGE BPX5 column (30 m×0.25 mm ID×0.25 μm film thickness) coated with 5% phenyl, 95% dimethylpolysiloxane. The oven temperature program consisted of an initial 50° C. hold for 2.0 min, followed by a 10° C./min ramp to 200° C. with no hold time before ramping to 240° C. at 5° C./min and maintaining that temperature for 5.0 min. The total run time was 30.0 min. The post-run temperature was set to 70° C. The MS transfer line temperature was maintained at 300° C. The mass spectrometer operated in scan mode with an electron ionization (EI) source at 230° C. and a quadrupole temperature of 150° C. Mass spectra were recorded over a range of m/z 50-600 with a solvent delay of 4.0 min. After, the sample injection mode was split-less with full scan mode within the range of 50 to 600 m/z, and helium gas was employed as the carrier gas with a flow rate of 1 mL/min based on a 10:1 split ratio.

### 1.5 Identification of Bioactive Compounds by Q-ToF-LCMS Analysis

The extract was analyzed using the 6200 series TOF/6500 series, version Q-TOFB.06.01, (B6172 SPI) iFunnel Q-ToF-LCMS (Quadrupole Time-of-Flight Liquid Chromatography Mass Spectrometry) (Agilent Technology, Santa Clara, Calif.) fitted with an electrospray interface and operating in positive and negative ion mode. 20 μg of each extract was prepared by dissolving it in 200 μl of methanol. The column (2.1×150 mm, 3.5 μm Agilent Zorbax Eclipse XDB-C18) was kept at 25° C., while the auto-sampler was kept at 4° C. Fresh 0.1% formic acid in water and 0.1% formic acid in acetonitrile mixtures were prepared for the mobile phases A and B, respectively. The flow rate was at 0.5 mL/min, the injection volume was at 1.0 μL, the run time was 25 min, and the recovery period was 5 min. Using an electrospray ion source in positive mode, a full scan MS analysis was done over the m/z 100-1000 range. The experiment was conducted using a capillary voltage of 3500 V. The data were processed using Agilent Mass Hunter Qualitative Analysis B.06.01 (Method: Metabolomics-2019.m). The compounds were discovered by comparisons and searching the METLIN database.

### 1.6 In Vivo Toxicity

### 1.6.1 Acute Toxicity

The acute toxicity test was carried out according to the Organization for Economic Cooperation and Development (OECD) guidelines for handling research animals (OECD 423 and OECD 407). A total of 84 male Mice weighing between 28 and 32 g were randomly divided into four groups of each sample (4 groups*7 mice in each group*3 samples). The group and dose for fruits were designed as G1=Normal control (only water), G2=1875 mg/kg bw, G3=3750 mg/kg bw, and G4=7500 mg/kg bw. The dose for leaves was designed as G1=Normal control (only water), G2=1250 mg/kg bw, G3=2500 mg/kg bw, G4=5000 mg/kg bw. The dose for seeds was designed as G1=Normal control (only water), G2=5 ml/kg bw, G3=10 ml/kg bw, G4=20 ml/kg bw. Mortality and general behavior were observed at 30 min, 1 h, 3 h, 6 h, 10 h, and 24 h after administration of extract on the starting day and then daily for a total of 2 weeks. The LD50 in this research was calculated. The biochemical and histological analyses were analyzed using blood and organs-liver, kidney, heart, lung, and brain.

### 1.6.2 Subacute Toxicity

The subacute toxicity assay was carried out for 4 weeks according to the OECD 407 guidelines (OECD 407, 2008). 84 male mice were randomly divided into four groups comprising 7 mice each (4 groups*7 mice in each group*3 sample=84 mice). Based on acute toxicity, the dose was selected for the fruits, leaves and seeds and administered via oral gavage for 28 days, whereas the control group was given distilled water to normal mice. The group and dose for fruits were designed as G1=Normal control (only water), G2=938 mg/kg bw, G3=1875 mg/kg bw, and G4=3750 mg/kg bw. The dose for leaves was designed as G1=Normal control (only water), G2=625 mg/kg bw, G3=1250 mg/kg bw, G4=2500 mg/kg bw. The dose for seeds was designed as G1=Normal control (only water), G2=2.5 ml/kg bw, G3=5 ml/kg bw, G4=10 ml/kg bw. Throughout the treatment, the toxicity indicators and abnormal behavior were monitored on a regular basis. The weight was measured on a weekly basis and recorded accordingly. All animals were anesthetized with pentobarbital at the end of the therapy, and blood samples were taken immediately for biochemical examination. The specific organs, namely, liver, lung, heart, brain, and kidney were collected for the histological examination.

### 1.7 In Vivo Anti-Diabetic

After toxicity analysis, different doses of noni fruits and leaves were administered in different groups based on the study plan. A total of 72 male mice were divided randomly into 6 groups, each group contained six mice (n=6) (6 groups×6 mice each group×2 samples=72 mice). Groups are represented as G1-Normal mice, G2-Noni treated (200 mg/kg bw) on normal mice: G3-Diabetic control group (STZ treated); G4-Drug control (Glibenclamide 5 mg/kg bw); G5-Noni treated (200 mg/kg bw) on diabetic mice; G6-Noni treated (400 mg/kg bw) on diabetic mice. Except for the normal control groups G1 and G2, treated groups (G3-G6) were intraperitoneally injected with low-dose STZ (100 mg/kg bw) for the development/induction of diabetes. The mice of all groups were subjected to overnight fasting after the manifestation of diabetes after 7 days of injection of STZ, followed by checking the glucose level of the blood withdrawn from the mouse tail. The glucose was recorded weekly using a glucometer (Accu check Performa, Mannheim, Germany). Biochemical parameters (total cholesterol, high-density lipoprotein cholesterol (HDL-cholesterol, HDL-C), low-density lipoprotein cholesterol (LDL-cholesterol, LDL-C), triglycerides, alanine aminotransferase (ALT), aspartate aminotransferase (AST) and alkaline phosphatase (ALP), urea and creatinine) were also analyzed from blood samples at the end of the study period. Moreover, morphological findings of liver, heart, kidney and pancreas were observed using a fluorescence microscope.

### 2. Experimental Findings

### 2.1 Yield

Soxhlet extraction produced higher yields of the crude extracts for the leaves (28.58%), fruits (25.90%), and seeds (31.26%) compared to the purified extracts of scCO₂ extraction yielding 12.25%, 10.23%, and 9.40% from leaves, fruits, and seeds, respectively (Table 2). The yield is higher in Soxhlet extraction due to the use of high temperatures that degrade the plant materials, resulting in unwanted products that are also impure. On the other hand, scCO₂ extraction extracted only specific compounds using green solvents with optimized conditions. As a result, scCO₂ extraction produced comparatively pure extract but lower yields because non-target compounds remain unextracted.

The noni fruits produce a powerful unpleasant odor that some people find bothersome. The fruit earns its ‘vomit fruit’ name because its strong pungent smell intensifies during ripening and reaches distances of up to five meters according to some reports. Noni fruit has bitter taste and strong rotten smell, so people typically mix it with other fruits during juicing to alter the sensory properties of commercial noni juice. The unripe Noni fruit appears dark green in color but the ripe fruit gives off a strong butyric acid smell similar to decayed matter. The juice production from Noni fruit requires ripe Noni fruit which has both unpleasant odor and bitter taste. Unlike other Noni extract available, we surprisingly found that our Noni scCO₂ extracts were pungent-free with reduced or eliminated unpleasant smell associated generally with Noni products. Our optimized CO₂ extraction process helped to free the extract from the unpleasant compounds. This is because CO₂ is a bleaching agent which is also a non-polar solvent. Therefore, scCO₂ extract contains the thermolabile, polar and non-polar compounds due to optimization of temperature, pressure, co-solvent ratios, time, and flow rate. In this condition, scCO₂ extract preserves the natural fragrance due to avoiding high temperature and fermentation. In addition, there are no preservatives or additives or organic solvents other than small amount of food-grade ethanol used in this extraction process.

TABLE 2
The percentage yield of Morinda citrifolia (noni) leaves, fruits,
and seeds powder using Soxhlet (Soxh) and scCO₂ extraction.

| | scCO₂ extraction | Soxhlet extraction |
|---|---|---|
| | yield (%) | yield (%) |
| Samples | (Mean ± SD) | (Mean ± SD) |
| Leaves | 12.25 ± 1.53 | 28.58 ± 2.41 |
| Fruits | 10.23 ± 1.19 | 25.90 ± 3.05 |
| Seeds | 9.40 ± 0.70 | 31.26 ± 4.15 |

### 2.2. Bioactivity

The total phenolic content (TPC) and total flavonoid content (TFC) were found to be slightly higher in Soxhlet extracts than scCO₂ extracts (Table 3), and this may be due to the extraction of other unwanted compounds, which may lead to overestimation of the results. Although scCO₂ extraction gives a lower TPC and TFC than the Soxhlet extraction, the extracts obtained from scCO₂ extraction are of better quality as it is a selective method of extraction that causes less damage to compounds and is less likely to cause contamination. These characteristics make scCO₂ extraction a more suitable technique than Soxhlet for any application in which pure extracts are needed.

TABLE 3
Total phenolic content (TPC) and total flavonoid content (TFC)
of Morinda citrifolia leaves, fruits, and seeds extracts.

| | TPC | TFC |
|---|---|---|
| | GAE (mg/g) | QE (mg/g) |
| Name of extracts | (Mean ± SD) | (Mean ± SD) |
| scCO₂-leaves | 32.76 ± 1.75 | 23.84 ± 1.93 |
| scCO₂-fruits | 25.27 ± 2.25 | 13.19 ± 1.37 |
| scCO₂-seeds | 21.11 ± 2.37 | 10.35 ± 0.98 |
| Soxh (Eth)-leaves | 45.26 ± 3.07 | 27.53 ± 1.74 |
| Soxh (Eth)-fruits | 33.01 ± 1.09 | 19.86 ± 1.38 |
| Soxh (Eth)-seeds | 30.30 ± 2.43 | 14.71 ± 0.82 |

Antioxidant capacity, measured by FRAP and DPPH assays, revealed that extracts obtained by scCO₂ extraction had better activity than the Soxhlet extracts in leaves, fruits and seeds (Table 4). In the case of leaves, the antioxidant capacity by FRAP was 20.32±1.07 mg AAE/g, and the IC₅₀ was 2.606±0.304 mg/ml by DPPH. On the other hand, Soxhlet extracts had lower antioxidant activity with the highest FRAP value of 10.16±1.69 mg AAE/g also found in the leaves and a much lower DPPH IC₅₀ of 5.413±0.541 mg/ml. The higher FRAP and DPPH values that were noticed in scCO₂ extracts as compared to Soxhlet extracts are ascribed to the specific extraction properties of supercritical carbon dioxide, which keep and concentrate bioactive compounds with antioxidant potential. On the other hand, Soxhlet extraction tends to destroy some compounds since they are subjected to high temperatures and solvents for a long period, thereby decreasing the antioxidant activity.

TABLE 4
FRAP and DPPH findings of Morinda citrifolia
leaves, fruits and seeds extracts.

| | FRAP | DPPH |
|---|---|---|
| | AAE (mg/gm) | IC₅₀ (mg/mL) |
| Name of extracts | (Mean ± SD) | (Mean ± SD) |
| scCO₂-leaves | 20.32 ± 1.07 | 2.606 ± 0.304 |
| scCO₂-fruits | 16.96 ± 1.17 | 5.281 ± 0.676 |
| scCO₂-seeds | 9.52 ± 1.20 | 4.312 ± 0.519 |
| Soxh (Eth)-leaves | 10.16 ± 1.69 | 5.413 ± 0.541 |
| Soxh (Eth)-fruits | 7.36 ± 1.19 | 8.064 ± 0.728 |
| Soxh (Eth)-seeds | 8.86 ± 1.39 | 9.973 ± 0.365 |

In the in vitro anti-diabetic analysis, the half maximal α-glucosidase inhibitory concentration (IC₅₀) was much better in scCO₂ extraction process of leaves, fruits and seeds than the Soxhlet extraction process (Table 5). Among these parts, scCO₂ extraction-fruits showed more than 2.5 times better effects than scCO₂-leaves and about two times better than the scCO₂-seeds. The least effective one was the scCO₂-leaves with IC₅₀ of 43.89 μg/ml (about 50 μg/mL). The better effect was found in scCO₂ extraction due to the extraction capability of thermolabile polar and non-polar compounds with good quality when using our optimized process. In contrast, thermolabile compounds degrade with high temperatures and prolonged extraction time in the Soxhlet extraction techniques. It is worth noting that our unique extraction process resulted in scCO₂ extracts with very strong α-glucosidase inhibition effects compared to the values reported in the literature. Such powerful α-glucosidase inhibitory effect makes our scCO₂ extracts good candidates as anti-diabetic remedies. They can be given to patients at practical amounts suitable as drug or supplement (dietary supplement, nutraceutical or equivalent term). In comparison, reported extraction processes in the literature did not have such strong α-glucosidase inhibitory effects, making them not practical to be given as drugs or supplements.

TABLE 5
α-glucosidase inhibitory activity of Morinda citrifolia
leaves, fruits and seeds extracts.

| | α-Glucosidase Inhibitory |
|---|---|
| | IC₅₀ (μg/mL) |
| Name of extracts | (Mean ± SD) |
| scCO₂-leaves | 43.89 ± 2.46 |
| scCO₂-fruits | 16.31 ± 1.73 |
| scCO₂-seeds | 36.74 ± 2.57 |
| Soxh (Eth)-leaves | 254.71 ± 8.79 |
| Soxh (Eth)-fruits | 136.89 ± 6.67 |
| Soxh (Eth)-seeds | 171.05 ± 9.12 |

### 2.3. Compounds Identification Using GC-MS

Compounds identified using GC-MS from Soxhlet extraction are presented in Table 6 (from leaves), Table 7 (from fruits) and Table 8 (from seeds). Compounds identified from scCO₂ extraction using GC-MS are presented in Table 9 (from leaves), Table 10 (from fruits) and Table 11 (from seeds). scCO₂ extract is much more effective than Soxhlet for the extraction of compounds, where it is able to identify 22 (Table 9) compounds compared to 9 (Table 6). In fruits, both methods give an equal number of compounds, which is 21 (Table 7 and Table 10). scCO₂ extract gives better results in seeds as it identifies 18 compounds (Table 11) as against 8 by Soxhlet (Table 8). In general, scCO₂ extraction is a better technique, especially for the leaves and seeds. It is also efficient in isolating more compounds since it is able to penetrate the sample matrix to a greater extent when operated under supercritical conditions without affecting heat-sensitive compounds. Soxhlet gave similar results for fruits, which could be because the solvent and heat conditions used were enough for these samples.

TABLE 6
Compounds identified from Soxh. (Eth)-leaves extract using GC-MS.

| RT | Area Pct | Identified compounds | Qual |
|---|---|---|---|
| 16.3287 | 3.4013 | Cyclononasiloxane, octadecamethyl- | 87 |
| 17.8362 | 3.8021 | Cyclodecasiloxane, eicosamethyl- | 80 |
| 18.5368 | 3.8218 | Hexadecanoic acid, methyl ester | 96 |
| 19.4376 | 4.8499 | Glaucine | 53 |
| 20.7011 | 6.5364 | 9,12-Octadecadienoic acid (Z,Z)-, methyl ester | 99 |
| 20.7762 | 2.72 | 11-Octadecenoic acid, methyl ester | 93 |
| 20.795 | 2.9419 | 7,10,13-Hexadecatrienoic acid, methyl ester | 70 |
| 22.878 | 4.0449 | Silane, [4-[1,2-bis[(trimethylsilyl)oxy]ethyl]-1,2- | 43 |
| | | phenylene]bis(oxy)]bis[trimethyl- | |
| 29.4648 | 4.8397 | 1,3,3-Trimethyl-1-(4′-methoxyphenyl)-6-methoxyindane | 44 |

TABLE 7
Compounds identified from Soxh (Eth)-fruits extract using GC-MS.

| RT | Area Pct | Identified compounds | Qual |
|---|---|---|---|
| 5.0504 | 0.6326 | Hexanoic acid, methyl ester | 78 |
| 5.3569 | 0.1355 | Benz[e]azulene-3,8-dione, 3a,4,6a,7,9,10,10a,10b- | 42 |
| | | octahydro-3a,10a-dihydroxy-5-(hydroxymethyl)-2,10- | |
| | | dimethyl-, (3a.alpha.,6a.alpha.,10.beta.,10a.beta., 10b.beta.)-(+)- | |
| 8.3844 | 1.2982 | Octanoic acid, methyl ester | 90 |
| 9.5354 | 1.3839 | Octanoic Acid | 72 |
| 11.8124 | 0.2799 | 2-Bromo-2-methylsuccinic acid, dimethyl ester | 22 |
| 12.7569 | 0.3842 | Perhydro-htx-2-one, 2-depentyl-, acetate ester | 22 |
| 14.6773 | 1.2277 | Cyclooctasiloxane, hexadecamethyl- | 58 |
| 16.3287 | 5.9601 | Cyclononasiloxane, octadecamethyl- | 83 |
| 17.8362 | 8.7931 | Cycloheptasiloxane, tetradecamethyl- | 49 |
| 18.5368 | 0.697 | Hexadecanoic acid, methyl ester | 94 |
| 19.1123 | 0.178 | 3-Oxo-18-nor-ent-ros-4-ene-15.beta., 16-acetonide | 43 |
| 19.35 | 0.5145 | Hexadecanoic acid, ethyl ester | 43 |
| 19.4376 | 10.5451 | Benzoic acid, 2,4-bis[(trimethylsilyl)oxy]-, trimethylsilyl | 55 |
| | | ester | |
| 20.7012 | 1.7368 | 8,11-Octadecadienoic acid, methyl ester | 99 |
| 20.7762 | 0.7733 | 9-Octadecenoic acid, methyl ester | 93 |
| 21.1328 | 11.2456 | Benzeneethanamine, N-[(pentafluorophenyl)methylene]-.beta.,3,4- | 51 |
| | | tris[(trimethylsilyl)oxy]- | |
| 21.5894 | 2.2127 | 9,12-Octadecadienoic acid (Z,Z)- | 89 |
| 21.6457 | 0.5381 | 11-Dodecen-1-ol trifluoroacetate | 50 |
| 26.1683 | 1.3863 | Cis-8-methyl-exo-tricyclo[5.2.1.0(2.6)]decane | 76 |
| 26.6375 | 13.1333 | 1,3,5,7,9,11-Hexaethylbicyclo[5.5.1]hexasiloxane | 44 |
| 29.296 | 10.4686 | Benzoic acid, 2,5-bis(trimethylsiloxy)-, trimethylsilyl ester | 30 |

TABLE 8
Compounds identified from Soxh.
(Eth)-seeds extract using GC-MS.

| RT | Area Pct | Identified compounds | Qual |
|---|---|---|---|
| 19.3872 | 24.2899 | Hexadecanoic acid, methyl ester | 98 |
| 20.0565 | 3.8696 | 3,5-Difluorobenzaldehyde | 51 |
| | | carbamoylhydrazone | |
| 20.7571 | 1.3315 | Hexadecanoic acid, 14-methyl-, methyl ester | 98 |
| 21.783 | 35.9466 | 8,11-Octadecadienoic acid, methyl ester | 99 |
| 21.8518 | 25.5217 | 11-Octadecenoic acid, methyl ester | 99 |
| 22.2021 | 5.3397 | Octadecanoic acid, methyl ester | 99 |
| 22.6087 | 1.9558 | Tridecanoic acid | 81 |
| 22.7275 | 1.7451 | 9,12-Octadecadienoic acid, ethyl ester | 99 |

TABLE 9
Compounds identified from scCO₂-leaves extract using GC-MS.

| RT | Area Pct | Identified compounds | Qual |
|---|---|---|---|
| 5.6822 | 0.3006 | Pentasiloxane, dodecamethyl- | 72 |
| 8.1468 | 0.153 | N,N-Dimethyl-N′-(10-propyl-10H-acridin-9-ylidene)- | 47 |
| | | benzene-1,4-diamine | |
| 8.1718 | 0.135 | Cyclopentasiloxane, decamethyl- | 47 |
| 10.5989 | 0.4579 | Cyclohexasiloxane, dodecamethyl- | 50 |
| 12.7507 | 0.4446 | Cycloheptasiloxane, tetradecamethyl- | 42 |
| 14.6836 | 2.2069 | Silane, [4-[1,2-bis [(trimethylsilyl)oxy]ethyl]-1,2- | 91 |
| | | phenylene]bis(oxy)]bis[trimethyl- | |
| 15.5844 | 0.2493 | 1-(Adamantyl)cyclohexene | 47 |
| 16.335 | 6.8084 | Cyclononasiloxane, octadecamethyl- | 87 |
| 17.4359 | 0.3164 | Spirohexan-4-one, 5,5-dimethyl- | 43 |
| 17.5673 | 0.6321 | 2-Pentadecanone, 6,10,14-trimethyl- | 45 |
| 17.7174 | 0.1263 | 2-Cyclopenten-1-one, 3-(1-methylethyl)- | 38 |
| 17.8363 | 7.9314 | Cyclodecasiloxane, eicosamethyl- | 76 |
| 17.9301 | 0.1126 | 4,8-Decadienal, 5,9-dimethyl- | 43 |
| 18.5431 | 2.4946 | Pentadecanoic acid, 14-methyl-, methyl ester | 97 |
| 19.3563 | 0.292 | Hexadecanoic acid, ethyl ester | 64 |
| 19.4439 | 8.3241 | Cyclooctasiloxane, hexadecamethyl- | 68 |
| 20.7075 | 1.5398 | 9,12-Octadecadienoic acid, methyl ester | 99 |
| 20.8075 | 3.1463 | 9,12,15-Octadecatrienoic acid, methyl ester, (Z,Z,Z)- | 98 |
| 20.9076 | 2.2704 | Phytol | 53 |
| 23.9978 | 6.7568 | Hexasiloxane, tetradecamethyl- | 58 |
| 28.2326 | 0.207 | Methyl (5-hydroxy-1H-benzimidazol-2-yl)carbamate | 38 |
| 29.3148 | 9.8318 | Benzeneethanamine, N-[(pentafluorophenyl)methylene]-.beta.,3,4- | 53 |
| | | tris[(trimethylsilyl)oxy]- | |

TABLE 10
Compounds identified from scCO₂-fruits extract using GC-MS.

| RT | Area Pct | Identified compounds | Qual |
|---|---|---|---|
| 9.4853 | 11.746 | Octanoic Acid | 90 |
| 13.545 | 0.5889 | p-Chloroaniline | 27 |
| 14.6835 | 0.4678 | Cyclooctasiloxane, hexadecamethyl- | 83 |
| 16.3348 | 1.7596 | Cyclononasiloxane, octadecamethyl- | 94 |
| 17.8424 | 2.3086 | Cyclodecasiloxane, eicosamethyl- | 87 |
| 18.5492 | 0.8369 | Hexadecanoic acid, methyl ester | 98 |
| 19.2373 | 2.2456 | n-Hexadecanoic acid | 95 |
| 19.3562 | 1.8375 | Hexadecanoic acid, ethyl ester | 80 |
| 20.7073 | 4.469 | 8,11-Octadecadienoic acid, methyl ester | 99 |
| 20.7824 | 1.0842 | 8-Octadecenoic acid, methyl ester, (E)- | 99 |
| 21.3829 | 4.1493 | 9,12-Octadecadienoic acid (Z,Z)- | 89 |
| 21.5956 | 8.1942 | Linoleic acid ethyl ester | 99 |
| 21.6581 | 1.7163 | Z,Z-3,13-Octadecedien-1-ol | 60 |
| 22.0084 | 0.3597 | Octadecanoic acid, ethyl ester | 83 |
| 22.7653 | 0.3132 | Z,E-7,11-Hexadecadien-1-yl acetate | 53 |
| 24.6732 | 3.4718 | Cyclopentasiloxane, decamethyl- | 35 |
| 25.3863 | 0.2503 | 9,12-Octadecadienoic acid (Z,Z)-, 2,3-dihydroxypropyl | 64 |
| | | ester | |
| 26.1995 | 26.1582 | Cyclododecyne | 84 |
| 26.4997 | 3.8052 | 1,5,9,13-Tetradecatetraene | 95 |
| 26.6561 | 5.6005 | Benzeneethanamine, N- | 90 |
| | | [(pentafluorophenyl)methylene]-.beta.,3,4- | |
| | | tris[(trimethylsilyl)oxy]- | |
| 29.9214 | 0.7408 | 3,4-Octadiene, 7-methyl- | 74 |

TABLE 11
Compounds identified from scCO₂-seeds extract using GC-MS.

| RT | Area Pct | Identified compounds | Qual |
|---|---|---|---|
| 9.4978 | 0.8219 | Octanoic Acid | 72 |
| 13.545 | 0.4232 | n-Caprylic acid isobutyl ester | 16 |
| 13.9016 | 0.2893 | Phenol, 2,4-bis(1,1-dimethylethyl)- | 70 |
| 16.3349 | 5.203 | Cyclononasiloxane, octadecamethyl- | 87 |
| 17.5672 | 0.2555 | 2-Pentadecanone, 6,10,14-trimethyl- | 83 |
| 17.8362 | 5.7685 | Cyclodecasiloxane, eicosamethyl- | 91 |
| 18.543 | 1.0956 | Pentadecanoic acid, 14-methyl-, methyl ester | 93 |
| 19.2811 | 2.1773 | n-Hexadecanoic acid | 91 |
| 19.4438 | 7.3205 | Cyclooctasiloxane, hexadecamethyl- | 80 |
| 20.7073 | 5.8429 | 8,11-Octadecadienoic acid, methyl ester | 99 |
| 20.7699 | 1.307 | 16-Octadecenoic acid, methyl ester | 99 |
| 21.4079 | 3.4241 | 9,12-Octadecadienoic acid (Z,Z)- | 95 |
| 21.5831 | 1.9842 | Linoleic acid ethyl ester | 99 |
| 22.8779 | 6.1901 | Benzeneethanamine, N-[(pentafluorophenyl)methylene]-.beta.,3,4- | 25 |
| | | tris[(trimethylsilyl)oxy]- | |
| 23.5723 | 2.8934 | 4-Cyclopropylmethylbenzonitrile | 30 |
| 23.9976 | 4.9893 | 4,25-Secoobscurinervan-4-ol, 15,16-dimethoxy-22- | 46 |
| | | methyl-, (4.beta.,22.alpha.)- | |
| 26.1807 | 23.7541 | 13-Tetradece-11-yn-1-ol | 90 |
| 26.4872 | 2.0104 | Acetonitrile, 2,2′-iminobis- | 22 |

### 2.4 Compounds Identification Using Q-ToF-LC-MS

Compounds identified using Q-ToF-LC-MS from Soxhlet extraction are presented in Table 12a and Table 12b (from leaves), Table 13a and Table 13b (from fruits) and Table 14a and Table 14b (from seeds). Compounds identified from scCO₂ extraction using Q-ToF-LC-MS are presented in Table 15a and Table 15b (from leaves), Table 16a and Table 16b (from fruits), and Table 17a and Table 17b (from seeds). A total of 39 compounds were identified in the scCO₂ leaves extract using LC-MS analysis in both positive and negative ionization modes (Table 15a and 15b), whereas 28 compounds were detected through Soxhlet extraction (Table 12a and 12b). For the fruits, 42 compounds were identified using scCO₂ extraction with both polarities (Table 16a and 16b), in contrast to 30 compounds found using Soxhlet extraction (Table 13a and 13b). Similarly, 40 compounds were identified in seeds through scCO₂ extract (Table 17a and 17b), while 30 compounds were determined from the Soxhlet seed extract (Table 14a and 14b). Specifically, the data shows that scCO₂ extract is able to bring out more LC-MS identifiable compounds than Soxhlet extraction. This can be explained by the benefits of using scCO₂ extraction, where CO₂ in the supercritical state is used as a solvent. scCO₂ extraction works at relatively lower temperatures and offers enhanced solvent diffusion hence preventing thermal decomposition of compounds and loss of volatility. Also, the density of supercritical CO₂ can be adjusted and this helps in the selective extraction of compounds with certain polar characteristics. On the other hand, Soxhlet extraction often involves the use of high temperatures and organic solvents which may lead to degradation of the compounds or non-specific extraction. Among the compounds, some novel compounds such as Monotropein, m-Coumaric Acid, Betulinic Acid, Ganoderol A, Robinetin 3-Rutinoside, Luteolin 5,3′-Dimethyl Ether, C16 Sphinganine, Luteolin 5,7,3′,4′-Tetramethyl Ether, Hypolaetin 8,3′-Dimethyl Ether, 1-Linoleoyl Glycerol, 9Z,12Z,15E-Octadecatrienoic Acid, Lamiide, Reboxetine, Methylgingerol identified first time from CNMI sample that showed anti-diabetic effects.

TABLE 12a
Compounds identified of Noni Soxh (Eth)-leaves extract
through Q-ToF-LCMS negative polarization technique.

| Name | Formula | m/z | Score | RT |
|---|---|---|---|---|
| 1,9-Dimethyluric acid | C₇H₈N₄O₃ | 195.0522 | 94.75 | 0.653 |
| Hypoxanthine | C₅H₄N₄O | 135.0311 | 97.13 | 0.668 |
| Monotropein | C₁₆H₂₂O₁₁ | 389.1086 | 97.63 | 1.054 |
| Furafylline | C₁₂H₁₂N₄O₃ | 259.0833 | 97.66 | 1.132 |
| Phlorisobutyrophenone | C₁₀H₁₂O₄ | 195.0666 | 97.17 | 1.132 |
| trans-2- | C₁₁H₁₁N₅O₄ | 276.0733 | 97.07 | 1.132 |
| [(Dimethylamino)methylimino]-5-[2- | | | | |
| (5-nitro-2-furyl)-vinyl]-1,3,4- | | | | |
| oxadiazole | | | | |
| Robinetin 3-rutinoside | C₂₇H₃₀O₁₆ | 609.146 | 99.57 | 8.747 |
| Luteolin 7-rhamnosyl(1−>6)galactoside | C₂₇H₃₀O₁₅ | 593.1518 | 99.02 | 9.08 |
| Nonic Acid | C₉H₁₆O₄ | 187.0979 | 98.98 | 9.63 |
| Tomentosic acid | C₃₀H₄₈O₆ | 503.3379 | 99.84 | 12.662 |
| Luteolin 5,3′-dimethyl ether | C₁₇H₁₄O₆ | 313.0723 | 98.52 | 13.65 |
| Dihydroalbocycline | C₁₈H₃₀O₄ | 345.1841 | 99.42 | 14.05 |
| Arjunolic acid | C₃₀H₄₈O₅ | 487.3429 | 99.12 | 14.595 |
| α-9(10)-EpODE | C₁₈H₃₀O₃ | 293.2128 | 98.98 | 15.062 |
| 9Z,12Z,15E-octadecatrienoic acid | C₁₈H₃₀O₂ | 277.2172 | 99.43 | 18.513 |

TABLE 12b
Compounds identified of Noni Soxh (Eth)-leaves extract
through Q-ToF-LCMS positive polarization technique.

| Name | Formula | m/z | Score | RT |
|---|---|---|---|---|
| Pyroglutamic acid | C₅H₇NO₃ | 130.0499 | 97.44 | 0.958 |
| Robinetin 3-rutinoside | C₂₇H₃₀O₁₆ | 611.1615 | 98.58 | 8.751 |
| Luteolin 7-rhamnosyl(1−>6)galactoside | C₂₇H₃₀O₁₅ | 595.165 | 96.38 | 9.087 |
| 11-hydroperoxy-12,13-epoxy-9- | C₁₈H₃₂O₅ | 346.2583 | 98.87 | 11.084 |
| octadecenoic acid | | | | |
| 1α,25-dihydroxy-26,27-dimethyl- | C₃₀H₄O₃ | 453.3365 | 98.06 | 14.42 |
| 20,21,22,22,23,23-hexadehydro-24a- | | | | |
| homocholecalciferol | | | | |
| 2-hexyl-decanoic acid | C₁₆H₃₂O₂ | 279.2297 | 97.49 | 15.885 |
| cis-9,10-Epoxystearic acid | C₁₈H₃₄O₃ | 321.2405 | 95.41 | 17.714 |
| 16Z-octadecenoic acid | C₁₈H₃₄O₂ | 305.2452 | 93.88 | 18.04 |
| Ganoderol A | C₃₀H₄₆O₂ | 439.3568 | 98.88 | 19.045 |
| 5β-Cholestane-3α-ol | C₂₇H₄₈O | 411.3607 | 95.56 | 19.046 |
| Betulinic Acid | C₃₀H₄₈O₃ | 457.3667 | 97.17 | 19.047 |
| Harderoporphyrin | C₃₅H₃₆N₄O₆ | 609.2711 | 98.86 | 19.364 |
| Pheophorbide a | C₃₅H₃₆N₄O₅ | 593.2769 | 97.84 | 20.39 |

TABLE 13a
Compounds identified of Noni Soxh (Eth)-fruits extract
through Q-ToF-LCMS negative polarization technique.

| Name | Formula | m/z | Score | RT |
|---|---|---|---|---|
| Theobromine | C₇H₈N₄O₂ | 179.0572 | 95.02 | 0.656 |
| 3,4-Dehydro-6-hydroxymellein | C₁₀H₈O₄ | 191.0357 | 95.76 | 9.021 |
| 4R-hydroxy-octanoic acid | C₈H₁₆O₃ | 159.1028 | 99.8 | 10.241 |
| 12-oxo-10Z-octadecenoic acid | C₁₈H₃₂O₃ | 295.2291 | 94.97 | 17.173 |
| 1-Linoleoyl Glycerol | C₂₁H₃₈O₄ | 389.2481 | 91.88 | 18.551 |
| 2-hexyl-decanoic acid | C₁₆H₃₂O₂ | 255.2341 | 95.35 | 20.29 |
| 16Z-octadecenoic acid | C₁₈H₃₄O₂ | 281.2492 | 98.63 | 20.411 |

TABLE 13b
Compounds identified of Noni Soxh (Eth)-fruits extract
through Q-ToF-LCMS positive polarization technique.

| Name | Formula | m/z | Score | RT |
|---|---|---|---|---|
| Panose | C₁₈H₃₂O₁₆ | 522.2027 | 95.55 | 0.668 |
| Monotropein | C₁₆H₂₂O₁₁ | 408.1503 | 98.54 | 1.057 |
| Veranisatin C | C₁₆H₂₀O₁₀ | 373.1122 | 94.39 | 1.057 |
| Thr Gln Tyr | C₁₈H₂₆N₄O₇ | 428.2135 | 96.62 | 6.557 |
| 1-O-Caffeoylquinic acid | C₁₆H₁₈O₉ | 355.1028 | 98.92 | 7.003 |
| 3-Methyl-3-butenyl apiosyl- | C₁₆H₂₈O₁₀ | 398.2027 | 98.64 | 7.369 |
| (1 −> 6)-glucoside | | | | |
| Scopolin | C₁₆H₁₈O₉ | 355.1017 | 95.27 | 7.429 |
| 1-(3-Methylbutanoyl)-6- | C₁₆H₂₈O₁₁ | 414.1974 | 98.57 | 7.442 |
| apiosylglucose | | | | |
| Prenyl apiosyl-(1 −> 6)- | C₁₆H₂₈O₁₀ | 398.2029 | 96.4 | 7.51 |
| glucoside | | | | |
| Robinetin 3-rutinoside | C₂₇H₃₀O₁₆ | 611.1603 | 97.81 | 8.749 |
| Linalool oxide D 3-[apiosyl- | C₂₁H₃₆O₁₁ | 482.2593 | 93.57 | 8.846 |
| (1 −> 6)-glucoside] | | | | |
| Paeonolide | C₂₀H₂₈O₁₂ | 478.1929 | 92.08 | 8.887 |
| Pirimicarb | C₁₁H₁₈N₄O₂ | 261.1321 | 97.53 | 9.053 |
| Val Arg | C₁₁H₂₃N₅O₃ | 296.1694 | 96.32 | 9.072 |
| (2R,3S)-2,3-Dimethylmalate | C₆H₁₀O₅ | 325.1128 | 94.51 | 9.151 |
| Bis-D-fructose 2′,1:2,1′- | C₁₂H₂₀O₁₀ | 325.1126 | 96.4 | 10.314 |
| dianhydride | | | | |
| C16 Sphinganine | C₁₆H₃₅NO₂ | 274.2736 | 99.04 | 12.166 |
| 4,5-Di-O-methyl-8- | C₂₂H₂₆O₆ | 387.1795 | 98.49 | 13.133 |
| prenylafzelechin-4beta-ol | | | | |
| 7-Hexadecen-1-ol | C₁₆H₃₂O | 263.2346 | 94.56 | 18.547 |
| 1-Linoleoyl Glycerol | C₂₁H₃₈O₄ | 377.2656 | 98.07 | 18.55 |
| Betulinic Acid | C₃₀H₄₈O₃ | 935.7104 | 98.18 | 19.047 |
| 7-Hexadecen-1-ol | C₁₆H₃₂O | 263.2348 | 92.12 | 19.431 |
| 3α,12α-Dihydroxy-5β-chol- | C₂₄H₃₈O₄ | 391.2836 | 95.51 | 21.315 |
| 9(11)-en-24-oic Acid | | | | |

TABLE 14a
Compounds identified of Noni Soxh (Eth)-seeds extract
through Q-ToF-LCMS negative polarization technique.

| Name | Formula | m/z | Score | RT |
|---|---|---|---|---|
| Acetylenedicarboxylate | C₄H₂O₄ | 112.9878 | 97.63 | 19.3 |
| 6E,9E-octadecadienoic acid | C₁₈H₃₂O₂ | 279.2341 | 85.64 | 19.425 |
| Prenyl arabinosyl-(1 −> 6)- | C₁₆H₂₈O₁₀ | 379.1604 | 76.31 | 19.443 |
| glucoside | | | | |
| Pro Lys Pro | C₁₆H₂₈N₄O₄ | 339.2026 | 80.31 | 19.464 |
| 2-hexyl-decanoic acid | C₁₆H₃₂O₂ | 255.2333 | 99.23 | 20.283 |
| 1,2,10-Trihydroxydihydro- | C₁₆H₃₀O₁₀ | 381.1767 | 75.2 | 20.42 |
| trans-linalyl oxide 7-O- | | | | |
| beta-D-glucopyranoside | | | | |

TABLE 14b
Compounds identified of Noni Soxh (Eth)-seeds extract
through Q-ToF-LCMS positive polarization technique.

| Name | Formula | m/z | Score | RT |
|---|---|---|---|---|
| D-(+)-Cellobiose | C₁₂H₂₂O₁₁ | 365.1054 | 93.48 | 0.657 |
| Nigerose (Sakebiose) | C₁₂H₂₂O₁₁ | 360.1507 | 96.28 | 0.668 |
| Monotropein | C₁₆H₂₂O₁₁ | 408.1496 | 99.38 | 0.789 |
| Vidarabine | C₁₀H₁₃N₅O₄ | 268.1036 | 94.98 | 1.057 |
| 1-O-Caffeoylquinic acid | C₁₆H₁₈O₉ | 355.1022 | 95.86 | 1.059 |
| Dihydroferulic acid 4-O- | C₁₆H₂₀O₁₀ | 373.1134 | 97.95 | 1.06 |
| glucuronide | | | | |
| Oleoside dimethyl ester | C₁₈H₂₆O₁₁ | 436.1808 | 98.98 | 3.189 |
| Apiosylglucosyl 4- | C₁₈H₂₄O₁₂ | 450.1612 | 98.79 | 7.077 |
| hydroxybenzoate | | | | |
| Wairol | C₁₇H₁₂O₆ | 313.0703 | 99.34 | 8.523 |
| Hypolaetin 8,3′-dimethyl ether | C₁₇H₁₄O₇ | 331.0813 | 91.54 | 8.834 |
| 1-Naphthyl β-D-glucuronide | C₁₆H₁₆O₇ | 321.0966 | 96.4 | 8.857 |
| Wightin | C₁₈H₁₆O₇ | 362.1234 | 98.88 | 9.392 |
| (2S)-5,6,7,3′,4′- | C₂₀H₂₂O₇ | 375.1433 | 98.37 | 9.532 |
| Pentamethoxyflavanone | | | | |
| 5,7-Dihydroxy-8,2′,6′- | C₁₈H₁₆O₇ | 345.0964 | 90.89 | 10.008 |
| trimethoxyflavone | | | | |
| 2′-Hydroxy-3,5,7,4′,5′- | C₂₀H₂₀O₈ | 389.1231 | 98.56 | 10.117 |
| pentamethoxyflavone | | | | |
| Phellodensin D | C₂₀H₂₀O₆ | 357.1334 | 98.18 | 10.922 |
| 3-Hydroxy-8,9- | C₁₇H₁₂O₆ | 313.0705 | 98.15 | 10.971 |
| dimethoxycoumestan | | | | |
| Luteolin 5,7,3′,4′-tetramethyl | C₁₉H₁₈O₆ | 343.1168 | 97.56 | 11.248 |
| ether | | | | |
| Eplerenone | C₂₄H₃₀O₆ | 415.2107 | 97.38 | 14.112 |
| Lucidumol A | C₃₀H₄₈O₄ | 473.3616 | 94.89 | 15.969 |
| (—)-Sanggenone K | C₃₀H₃₂O₆ | 489.2269 | 97.03 | 18.143 |
| 7-Hexadecen-1-ol | C₁₆H₃₂O | 263.2343 | 90.96 | 18.547 |
| Ganoderol A | C₃₀H₄₆O₂ | 439.3567 | 97.37 | 19.032 |
| Betulinic Acid | C₃₀H₄₈O₃ | 935.711 | 96.25 | 19.037 |

TABLE 15a
Compounds identified of Noni scCO₂ leaves extract
through Q-ToF-LCMS negative polarization technique.

| Name | Formula | m/z | Score | RT |
|---|---|---|---|---|
| Nonic Acid | C₉H₁₆O₄ | 187.098 | 98.67 | 9.646 |
| 7-hydroxy pelargonic acid | C₉H₁₈O₃ | 173.1186 | 99 | 9.734 |
| 11-hydroperoxy-12,13-epoxy-9- | C₁₈H₃₂O₅ | 327.2184 | 98.36 | 11.115 |
| octadecenoic acid | | | | |
| 5,8,12-trihydroxy-9- | C₁₈H₃₄O₅ | 329.2342 | 97.25 | 11.514 |
| octadecenoic acid | | | | |
| Methylgingerol | C₁₈H₂₈O₄ | 307.1926 | 94.5 | 12.633 |
| Corrinoid | C₁₉H₂₂N₄ | 305.177 | 93.77 | 13.36 |
| Luteolin 5,3′-dimethyl ether | C₁₇H₁₄O₆ | 313.0726 | 95.6 | 13.661 |
| Dihydroalbocycline | C₁₈H₃₀O₄ | 309.2075 | 84.7 | 13.879 |
| 5Z,8Z,11Z,14Z- | C₁₈H₂₈O₂ | 275.2026 | 92.21 | 15.068 |
| octadecatetraenoic acid | | | | |
| cyasterone | C₂₉H₄₄O₈ | 519.2966 | 84.43 | 18.149 |
| 9Z,12Z,15E-octadecatrienoic | C₁₈H₃₀O₂ | 277.2166 | 97.31 | 18.507 |
| acid | | | | |

TABLE 15b
Compounds identified of Noni scCO₂ leaves extract
through Q-ToF-LCMS positive polarization technique.

| Name | Formula | m/z | Score | RT |
|---|---|---|---|---|
| 3,4,5-Trimethoxyphenyl | C₁₁H₁₄O₅ | 227.0904 | 94.03 | 7.237 |
| acetate | | | | |
| 3-oxo-tridecanoic acid | C₁₃H₂₄O₃ | 229.1787 | 94.27 | 7.961 |
| 4-(2-hydroxypropoxy)-3,5- | C₁₁H₁₆O₃ | 197.1166 | 97.33 | 9.283 |
| dimethyl-Phenol | | | | |
| 10-Tridecynoic acid | C₁₃H₂₂O₂ | 211.1685 | 97.13 | 10.595 |
| 11-hydroperoxy-12,13-epoxy- | C₁₈H₃₂O₅ | 346.2578 | 96.38 | 11.105 |
| 9-octadecenoic acid | | | | |
| 3-(2,3- | C₉H₁₀O₄ | 205.0479 | 92.16 | 11.271 |
| Dihydroxyphenyl)propanoate | | | | |
| Methylgingerol | C₁₈H₂₈O₄ | 309.2051 | 97.2 | 12.632 |
| Methoprene acid | C₁₆H₂₈O₃ | 291.194 | 93.5 | 12.635 |
| Burseran | C₂₂H₂₆O₆ | 387.1788 | 95.03 | 13.15 |
| Latanoprost Lactol | C₁₈H₂₆O₄ | 307.1894 | 96 | 13.363 |
| 5Z,8Z,11Z,14Z- | C₁₈H₂₈O₂ | 277.215 | 96.52 | 15.077 |
| octadecatetraenoic acid | | | | |
| 10E,12Z-Tetradecadienyl | C₁₆H₂₈O₂ | 275.1986 | 96.68 | 15.677 |
| acetate | | | | |
| 9,10-EOT | C₁₈H₂₈O₃ | 293.2097 | 91.59 | 15.678 |
| 2-hexyl-decanoic acid | C₁₆H₃₂O₂ | 279.2297 | 99.06 | 15.885 |
| α-9(10)-EpODE | C₁₈H₃₀O₃ | 295.2257 | 95.29 | 16.287 |
| 8E-Tetradecenyl acetate | C₁₆H₃₀O₂ | 277.2137 | 96.83 | 16.489 |
| Emmotin A | C₁₆H₂₂O₄ | 279.1582 | 96.3 | 16.95 |
| MG(0:0/18:3(6Z,9Z,12Z)/0:0) | C₂₁H₃₆O₄ | 353.2674 | 95.07 | 17.503 |
| 5-Androstene-3b,16b,17a-triol | C₁₉H₃₀O₃ | 307.2262 | 98.64 | 17.826 |
| 11(S)-HETE | C₂₀H₃₂O₃ | 321.2417 | 95.53 | 18.471 |
| 9Z,12Z,15E-octadecatrienoic | C₁₈H₃₀O₂ | 279.2309 | 96.19 | 18.503 |
| acid | | | | |
| 1-Linoleoyl Glycerol | C₂₁H₃₈O₄ | 377.2661 | 97.56 | 18.528 |
| Bryononic acid | C₃₀H₄₆O₃ | 455.3513 | 94.15 | 18.554 |
| Ganoderol A | C₃₀H₄₆O₂ | 439.3562 | 90.97 | 18.998 |
| 2alpha-(Hydroxymethyl)- | C₂₀H₃₄O₃ | 323.2565 | 90.19 | 19.229 |
| 5alpha-androstane- | | | | |
| 3beta,17beta-diol | | | | |
| Pheophorbide a | C₃₅H₃₆N₄O₅ | 593.2765 | 99.21 | 20.016 |
| Haplophytine | C₃₇H₄₀N₄O₇ | 653.2984 | 96.65 | 20.993 |
| N-Hexadecanoylpyrrolidine | C₂₀H₃₉NO | 310.3107 | 98.81 | 21.358 |

TABLE 16a
Compounds identified of Noni scCO₂ fruits extract
through Q-ToF-LCMS negative polarization technique.

| Name | Formula | m/z | Score | RT |
|---|---|---|---|---|
| Hypoxanthine | C₅H₄N₄O | 135.0313 | 96.21 | 0.67 |
| Dyphylline | C₁₀H₁₄N₄O₄ | 253.0942 | 96.55 | 0.923 |
| 7-Hydroxyethyltheophylline | C₉H₁₂N₄O₃ | 259.06 | 95.83 | 0.957 |
| Monotropein | C₁₆H₂₂O₁₁ | 389.1105 | 96.49 | 1.057 |
| 3-Furanmethanol glucoside | C₁₁H₁₆O₇ | 259.0826 | 99.02 | 1.14 |
| 2-(beta-D-Glucosyl)-sn- | C₉H₁₈O₈ | 253.0934 | 98.53 | 1.147 |
| glycerol | | | | |
| Lamiide | C₁₇H₂₆O₁₂ | 421.1357 | 98.58 | 1.221 |
| Dyphylline | C₁₀H₁₄N₄O₄ | 253.0939 | 97.79 | 1.4 |
| a-L-Fucopyranosyl-(1 −> 2)- | C₁₇H₃₀O₁₄ | 457.156 | 99.6 | 2.603 |
| b-D-galactopyranosyl-(1 −> | | | | |
| 2)-D-xylose | | | | |
| Levoglucosan | C₆H₁₀O₅ | 161.0458 | 99.54 | 2.67 |
| Apiosylglucosyl 4- | C₁₈H₂₄O₁₂ | 431.1211 | 96.15 | 7.073 |
| hydroxybenzoate | | | | |
| Prenyl arabinosyl-(1 −> | C₁₆H₂₈O₁₀ | 415.1383 | 96.63 | 7.499 |
| 6)-glucoside | | | | |
| Robinetin 3-rutinoside | C₂₇H₃₀O₁₆ | 609.1466 | 99.12 | 8.746 |
| O-Benzyl-L-Serine | C₁₀H₁₃NO₃ | 194.0821 | 99.65 | 14.348 |
| 6E,9E-octadecadienoic acid | C₁₈H₃₂O₂ | 279.2327 | 99.49 | 19.436 |

TABLE 16b
Compounds identified of Noni scCO₂ fruits extract
through Q-ToF-LCMS positive polarization technique.

| Name | Formula | m/z | Score | RT |
|---|---|---|---|---|
| Kojic Acid | C₆H₆O₄ | 143.0333 | 97.99 | 1.213 |
| (E)-2-Methyl-2-buten-1-ol | C₁₁H₂₀O₆ | 266.1591 | 96.15 | 6.668 |
| O-beta-D-Glucopyranoside | | | | |
| Pirimicarb | C₁₁H₁₈N₄O₂ | 261.132 | 98.82 | 8.55 |
| Val Arg | C₁₁H₂₃N₅O₃ | 296.1696 | 98.93 | 8.854 |
| 3,4-Dehydro-6- | C₁₀H₈O₄ | 193.049 | 98.49 | 9.02 |
| hydroxymellein | | | | |
| Glycerol tripropanoate | C₁₂H₂₀O₆ | 261.1327 | 98.5 | 9.106 |
| 1-(beta-D- | C₁₄H₂₆O₇ | 324.2009 | 97.4 | 10.732 |
| Glucopyranosyloxy)- | | | | |
| 3-octanone | | | | |
| C16 Sphinganine | C₁₆H₃₅NO₂ | 274.2732 | 96.35 | 12.193 |
| 4,5-Di-O-methyl-8- | C₂₂H₂₆O₆ | 387.1793 | 96.89 | 13.147 |
| prenylafzelechin-4beta-ol | | | | |
| beta-Zearalanol | C₁₈H₂₆O₅ | 323.1841 | 95.27 | 13.297 |
| 3-Hydroxy-8,9- | C₁₆H₁₀O₅ | 283.0588 | 91.27 | 13.656 |
| methylenedioxypterocarp- | | | | |
| 6a-ene | | | | |
| Ceriporic acid C | C₂₁H₃₆O₄ | 353.2674 | 94.14 | 14.886 |
| 8E-Tetradecenyl acetate | C₁₆H₃₀O₂ | 277.2148 | 92 | 15.188 |
| 9Z,12Z,15E-octadecatrienoic | C₁₈H₃₀O₂ | 279.2305 | 91.96 | 15.877 |
| acid | | | | |
| α-9(10)-EpODE | C₁₈H₃₀O₃ | 295.226 | 96.54 | 16.488 |
| Emmotin A | C₁₆H₂₂O₄ | 279.1588 | 99.06 | 16.951 |
| 2-hexyl-decanoic acid | C₁₆H₃₂O₂ | 279.2291 | 98.66 | 17.121 |
| 7-Hexadecen-1-ol | C₁₆H₃₂O | 263.234 | 94.78 | 18.292 |
| 1-Linoleoyl Glycerol | C₂₁H₃₈O₄ | 355.285 | 98.93 | 18.546 |
| Bryononic acid | C₃₀H₄₆O₃ | 455.3516 | 97.61 | 18.585 |
| Ganoderol A | C₃₀H₄₆O₂ | 439.3577 | 98.69 | 19.029 |
| Betulinic Acid | C₃₀H₄₈O₃ | 935.7112 | 95.42 | 19.035 |
| Dehydroconicasterol | C₂₉H₄₆O | 411.3619 | 95.96 | 19.036 |
| 6E,9E-octadecadienoic acid | C₁₈H₃₂O₂ | 281.2473 | 99.48 | 19.422 |
| Heneicosanedioic acid | C₂₁H₄₀O₄ | 357.2999 | 98.74 | 19.436 |
| 10-oxo-nonadecanoic acid | C₁₉H₃₆O₃ | 313.2732 | 97.42 | 19.486 |
| 1-Monopalmitin | C₁₉H₃₈O₄ | 353.2675 | 97.04 | 19.488 |

TABLE 17a
Compounds identified of Noni scCO₂ seeds extract through
Q-ToF-LCMS negative polarization technique.

| Name | Formula | m/z | Score | RT |
|---|---|---|---|---|
| L-Arabinonic acid | C₅H₁₀O₆ | 165.0406 | 99.36 | 0.654 |
| Nigerose (Sakebiose) | C₁₂H₂₂O₁₁ | 341.1096 | 96.12 | 0.679 |
| Monotropein | C₁₆H₂₂O₁₁ | 389.1093 | 99.2 | 1.726 |
| p-Salicylic acid | C₇H₆O₃ | 137.0243 | 99.3 | 5.723 |
| Dumosol | C₁₇H₁₄O₈ | 345.0614 | 98.94 | 7.023 |
| Apiosylglucosyl 4- | C₁₈H₂₄O₁₂ | 431.1194 | 99.06 | 7.07 |
| hydroxybenzoate | | | | |
| 4-Hydroxyphenylpyruvic acid | C₉H₈O₄ | 179.0351 | 99.55 | 7.77 |
| beta-D-Galactopyranosyl- | C₁₈H₃₃NO₁₅ | 502.178 | 99.57 | 8.217 |
| (1 −> 4)-2-amino-2-deoxy- | | | | |
| beta-D-glucopyranosyl- | | | | |
| (1 −> 6)-D-mannose | | | | |
| m-Coumaric acid | C₉H₈O₃ | 163.0402 | 97.52 | 8.439 |
| Hypolaetin 8,3′-dimethyl ether | C₁₇H₁₄O₇ | 329.0671 | 99.04 | 8.522 |
| Luteolin 5,3′-dimethyl ether | C₁₇H₁₄O₆ | 313.0723 | 97.36 | 8.837 |
| Mesquitol-4alpha-ol 8-methyl | C₁₆H₁₆O₇ | 319.0824 | 99.27 | 8.853 |
| ether | | | | |
| 5,6,5′-Trihydroxy-3,7,2′,4′- | C₁₉H₁₈O₉ | 389.0881 | 99.5 | 9.39 |
| tetramethoxyflavone | | | | |
| 2′,4′-Dihydroxy-2,3′,6′- | C₁₈H₁₈O₆ | 329.1027 | 98 | 9.665 |
| trimethoxychalcone | | | | |
| 8-Methoxycirsilineol | C₁₉H₁₈O₈ | 373.0934 | 98.03 | 9.689 |
| 2-O-(beta-D- | C₂₁H₃₈O₁₄ | 513.2185 | 97.04 | 9.908 |
| galactopyranosyl-(1 −> | | | | |
| 6)-beta-D-galactopyranosyl) | | | | |
| 2S,3R-dihydroxynonanoic | | | | |
| acid | | | | |
| Corymbosin | C₁₉H₁₈O₇ | 357.0982 | 99.69 | 10.316 |

TABLE 17b
Compounds identified of Noni scCO₂ seeds extract through
Q-ToF-LCMS positive polarization technique.

| Name | Formula | m/z | Score | RT |
|---|---|---|---|---|
| 3,4-Dehydro-6- | C₁₀H₈O₄ | 193.0487 | 96.75 | 9.022 |
| hydroxymellein | | | | |
| Phellodensin D | C₂₀H₂₀O₆ | 357.1325 | 96.13 | 10.941 |
| C16 Sphinganine | C₁₆H₃₅NO₂ | 274.2729 | 94.49 | 12.196 |
| Burseran | C₂₂H₂₆O₆ | 387.1784 | 94.83 | 13.147 |
| Oxprenolol | C₁₅H₂₃NO₃ | 288.1573 | 97.46 | 13.556 |
| Morphine | C₁₇H₁₉NO₃ | 286.1426 | 94.76 | 13.937 |
| Reboxetine | C₁₉H₂₃NO₃ | 314.1737 | 92.65 | 14.653 |
| MG(0:0/18:3(6Z,9Z,12Z)/0:0) | C₂₁H₃₆O₄ | 353.2681 | 93.28 | 14.843 |
| Homodihydrocapsaicin | C₁₉H₃₁NO₃ | 344.2197 | 98.48 | 16.643 |
| Emmotin A | C₁₆H₂₂O₄ | 279.158 | 96.46 | 16.949 |
| 2-hexyl-decanoic acid | C₁₆H₃₂O₂ | 279.2291 | 98.63 | 17.123 |
| 1-Linoleoyl Glycerol | C₂₁H₃₈O₄ | 355.2831 | 94.62 | 18.549 |
| Ganoderol A | C₃₀H₄₆O₂ | 439.357 | 98.91 | 19.024 |
| Betulinic Acid | C₃₀H₄₈O₃ | 935.7116 | 98.3 | 19.034 |
| 6E,9E-octadecadienoic acid | C₁₈H₃₂O₂ | 281.2467 | 97.17 | 19.424 |
| Heneicosanedioic acid | C₂₁H₄₀O₄ | 379.2809 | 90.57 | 19.439 |
| 1-Monopalmitin | C₁₉H₃₈O₄ | 353.2663 | 99.58 | 19.49 |
| Heneicosanedioic acid | C₂₁H₄₀O₄ | 379.2808 | 95.33 | 19.729 |
| 14,15-EE-5(Z)-E | C₂₀H₃₆O₃ | 325.2741 | 90.86 | 19.948 |
| 2,4,12-Octadecatrienoic acid | C₂₂H₃₉NO | 334.3103 | 97.56 | 20.261 |
| isobutylamide | | | | |
| N-Hexadecanoylpyrrolidine | C₂₀H₃₉NO | 310.3093 | 94.95 | 21.381 |
| 2,4,14-Eicosatrienoic acid | C₂₄H₄₃NO | 362.3415 | 97.83 | 21.477 |
| isobutylamide | | | | |
| Pipercitine | C₂₃H₄₃NO | 350.3419 | 95.13 | 24.294 |

### 2.5 In Vivo Acute and Subacute Toxicity

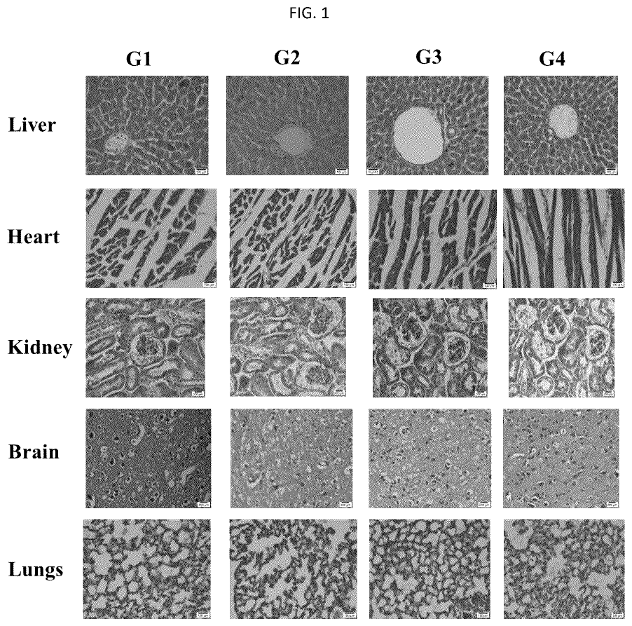

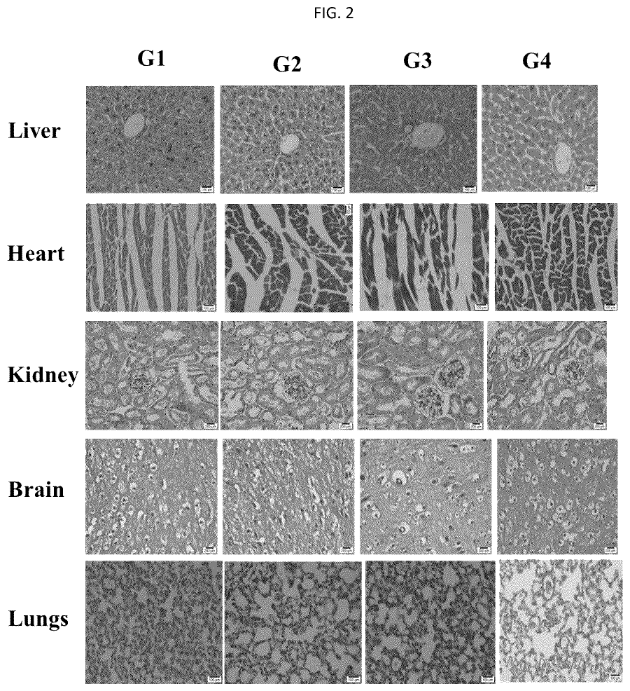

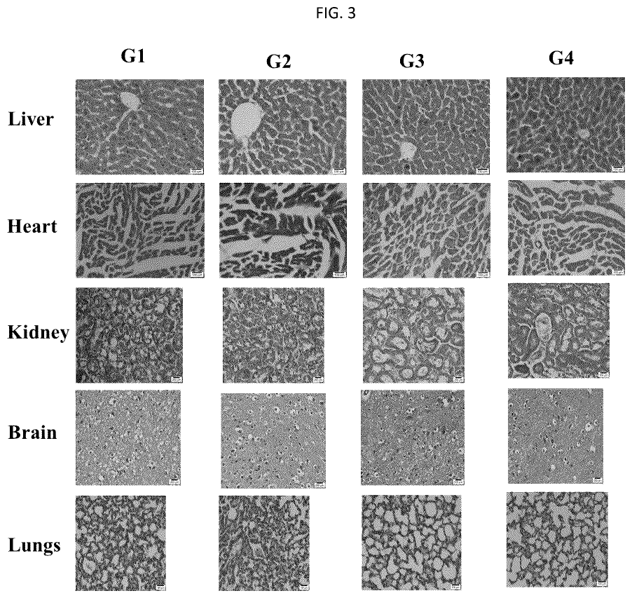

The acute toxicity result was analyzed in male mice according to the Organization for Economic Cooperation and Development (OECD) guideline. The acute toxicity results showed that there were no symptoms of general behavioral changes and no mortality until the end of the study in any of the groups (Table 18, Table 19, Table 20). The body weight increased gradually through the study period. Biochemical parameter findings were also normal compared to the normal control group (Table 21, Table 22, Table 23). The morphological findings of the liver, heart, kidney, lungs, and brain did not reveal any abnormalities (FIG. 1, FIG. 2, FIG. 3).

Based on the result, LD50 for noni leaves, fruits and seeds was >5000 mg/kg bw, >7500 mg/kg bw and >20 ml/kg bw, respectively. These doses are more than 25 times of the lowest dose used in the anti-diabetic study.

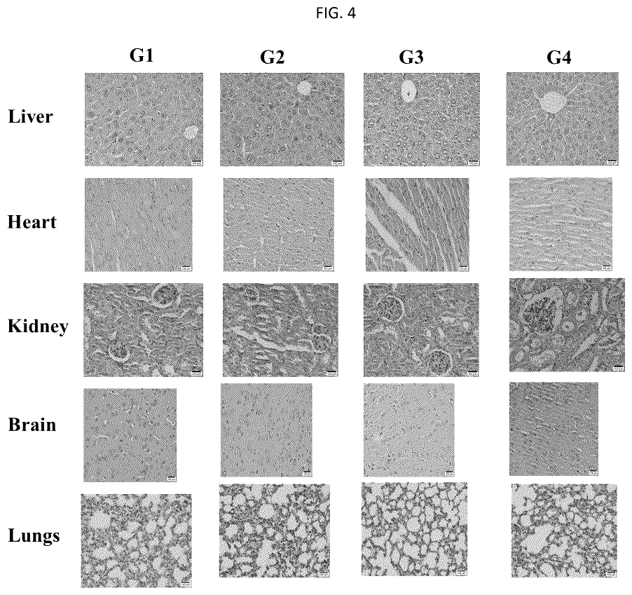

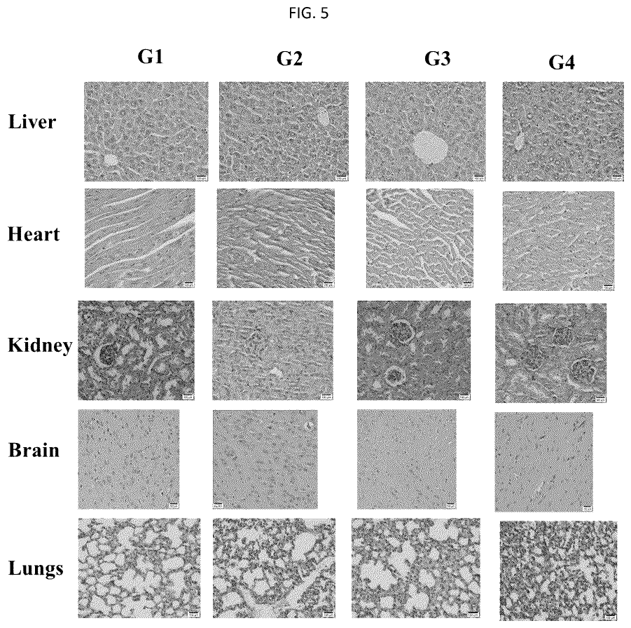

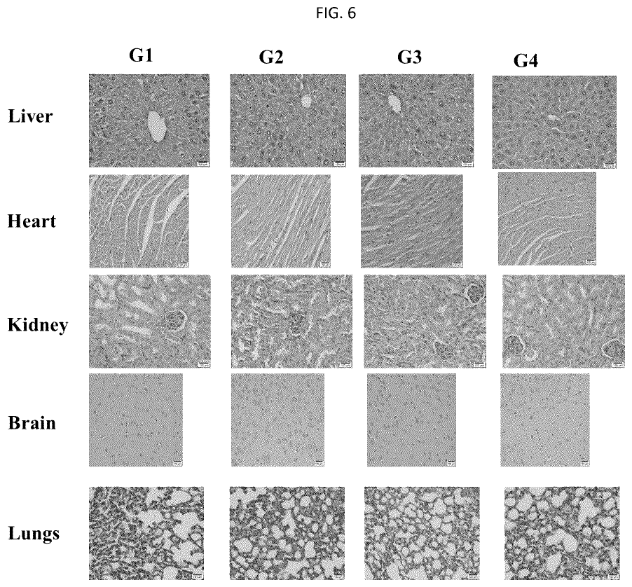

Based on the acute toxicity findings, we analyzed the sub-acute toxicity according to the OECD guideline 407. No toxicity was noticed in biochemical parameters (lipid profile, liver function, creatinine, and urea) (Table 24, Table 25, Table 26). In addition, morphological findings of liver, heart, kidney, lung, and brain of different dose-treated groups were found to have normal structure similar to that of the normal standard group (FIG. 4, FIG. 5, FIG. 6). The doses used in the sub-acute study are more than 10 times of the lowest dose used in the anti-diabetic study.

TABLE 18
Effect of Noni fruits extract on general behaviors in the acute toxicity study.

| | 30 min | | | | 1 hr | | | | 3 hr | | | | 6 hr | | | | 10 hr | | | | 24 hr | | | | 14 days | | | |
|---|---|---|---|---|---|---|---|---|---|---|---|---|---|---|---|---|---|---|---|---|---|---|---|---|---|---|---|---|
| Characteristics | G1 | G2 | G3 | G4 | G1 | G2 | G3 | G4 | G1 | G2 | G3 | G4 | G1 | G2 | G3 | G4 | G1 | G2 | G3 | G4 | G1 | G2 | G3 | G4 | G1 | G2 | G3 | G4 |
| Itching | N | N | N | N | N | N | N | N | N | N | N | N | N | N | N | N | N | N | N | N | N | N | N | N | N | N | N | N |
| Respiration | N | N | N | N | N | N | N | N | N | N | N | N | N | N | N | N | N | N | N | N | N | N | N | N | N | N | N | N |
| Feces | N | N | N | N | N | N | N | N | N | N | N | N | N | N | N | N | N | N | N | N | N | N | N | N | N | N | N | N |
| consistency | | | | | | | | | | | | | | | | | | | | | | | | | | | | |
| Convulsions | N | N | N | N | N | N | N | N | N | N | N | N | N | N | N | N | N | N | N | N | N | N | N | N | N | N | N | N |
| & tremors | | | | | | | | | | | | | | | | | | | | | | | | | | | | |
| Sleep | N | N | N | N | N | N | N | N | N | N | N | N | N | N | N | N | N | N | N | N | N | N | N | N | N | N | N | N |
| Coma | N | N | N | N | N | N | N | N | N | N | N | N | N | N | N | N | N | N | N | N | N | N | N | N | N | N | N | N |
| Salivation | N | N | N | N | N | N | N | N | N | N | N | N | N | N | N | N | N | N | N | N | N | N | N | N | N | N | N | N |
| Eyes | N | N | N | N | N | N | N | N | N | N | N | N | N | N | N | N | N | N | N | N | N | N | N | N | N | N | N | N |
| Mortality | — | — | — | — | — | — | — | — | — | — | — | — | — | — | — | — | — | — | — | — | — | — | — | — | — | — | — | — |

G1: Normal control (only water), G2 = 1875 mg/kg bw, G3 = 3750 mg/kg bw, G4 = 7500 mg/kg bw, N = Normal, — = Not found.

TABLE 19
Effect of Noni leaves extract on general behaviors in the acute toxicity study.

| | 30 min | | | | 1 hr | | | | 3 hr | | | | 6 hr | | | | 10 hr | | | | 24 hr | | | | 14 days | | | |
|---|---|---|---|---|---|---|---|---|---|---|---|---|---|---|---|---|---|---|---|---|---|---|---|---|---|---|---|---|
| Characteristics | G1 | G2 | G3 | G4 | G1 | G2 | G3 | G4 | G1 | G2 | G3 | G4 | G1 | G2 | G3 | G4 | G1 | G2 | G3 | G4 | G1 | G2 | G3 | G4 | G1 | G2 | G3 | G4 |
| Itching | N | O | O | N | N | N | N | N | N | N | N | N | N | N | N | N | N | N | N | N | N | N | N | N | N | N | N | N |
| Respiration | N | N | N | N | N | N | N | N | N | N | N | N | N | N | N | N | N | N | N | N | N | N | N | N | N | N | N | N |
| Feces | N | N | N | N | N | N | N | N | N | N | N | N | N | N | N | N | N | N | N | N | N | N | N | N | N | N | N | N |
| consistency | | | | | | | | | | | | | | | | | | | | | | | | | | | | |
| Convulsions | N | O | N | O | N | N | N | N | N | N | N | N | N | N | N | N | N | N | N | N | N | N | N | N | N | N | N | N |
| & tremors | | | | | | | | | | | | | | | | | | | | | | | | | | | | |
| Sleep | N | N | N | N | N | N | N | N | N | N | N | N | N | N | N | N | N | N | N | N | N | N | N | N | N | N | N | N |
| Coma | N | N | N | N | N | N | N | N | N | N | N | N | N | N | N | N | N | N | N | N | N | N | N | N | N | N | N | N |
| Salivation | N | N | N | N | N | N | N | N | N | N | N | N | N | N | N | N | N | N | N | N | N | N | N | N | N | N | N | N |
| Eyes | N | N | N | N | N | N | N | N | N | N | N | N | N | N | N | N | N | N | N | N | N | N | N | N | N | N | N | N |
| Mortality | — | — | — | — | — | — | — | — | — | — | — | — | — | — | — | — | — | — | — | — | — | — | — | — | — | — | — | — |

G1 = Normal control (only water), G2 = 1250 mg/kg bw, G3 = 2500 mg/kg bw, G4 = 5000 mg/kg bw, N = Normal, O = Observed, — = Not found.

TABLE 20
Effect of Noni seeds extract on general behaviors in the acute toxicity study.

| | 30 min | | | | 1 hr | | | | 3 hr | | | | 6 hr | | | | 10 hr | | | | 24 hr | | | | 14 days | | | |
|---|---|---|---|---|---|---|---|---|---|---|---|---|---|---|---|---|---|---|---|---|---|---|---|---|---|---|---|---|
| Characteristics | G1 | G2 | G3 | G4 | G1 | G2 | G3 | G4 | G1 | G2 | G3 | G4 | G1 | G2 | G3 | G4 | G1 | G2 | G3 | G4 | G1 | G2 | G3 | G4 | G1 | G2 | G3 | G4 |
| Itching | N | N | N | N | N | N | N | N | N | N | N | N | N | N | N | N | N | N | N | N | N | N | N | N | N | N | N | N |
| Respiration | N | N | N | N | N | N | N | N | N | N | N | N | N | N | N | N | N | N | N | N | N | N | N | N | N | N | N | N |
| Feces | N | N | N | N | N | N | N | N | N | N | N | N | N | N | N | N | N | N | N | N | N | N | N | N | N | N | N | N |
| consistency | | | | | | | | | | | | | | | | | | | | | | | | | | | | |
| Convulsions | N | N | N | N | N | N | N | N | N | N | N | N | N | N | N | N | N | N | N | N | N | N | N | N | N | N | N | N |
| & tremors | | | | | | | | | | | | | | | | | | | | | | | | | | | | |
| Sleep | N | N | N | N | N | N | N | N | N | N | N | N | N | N | N | N | N | N | N | N | N | N | N | N | N | N | N | N |
| Coma | N | N | N | N | N | N | N | N | N | N | N | N | N | N | N | N | N | N | N | N | N | N | N | N | N | N | N | N |
| Salivation | N | N | N | N | N | N | N | N | N | N | N | N | N | N | N | N | N | N | N | N | N | N | N | N | N | N | N | N |
| Eyes | N | N | N | N | N | N | N | N | N | N | N | N | N | N | N | N | N | N | N | N | N | N | N | N | N | N | N | N |
| Mortality | — | — | — | — | — | — | — | — | — | — | — | — | — | — | — | — | — | — | — | — | — | — | — | — | — | — | — | — |

G1 = Normal control (only water), G2 = 5 mL/kg bw, G3 = 10 mL/kg bw, G4 = 20 mL/kg bw, N = Normal, — = Not found.

TABLE 21
Effect of Noni fruit extract on biochemical analysis in the acute toxicity study.

| | G1 | G2 | G3 | G4 |
|---|---|---|---|---|
| Parameters | Mean ± SD | Mean ± SD | Mean ± SD | Mean ± SD |
| ALT (U/mL) | 28.4 ± 2.36 | 30.7 ± 3.05 | 27.2 ± 2.14 | 34.6 ± 3.52 |
| AST (U/mL) | 22.5 ± 1.65 | 25.2 ± 2.36 | 24 ± 2.18 | 33.8 ± 3.05 |
| ALP (U/mL) | 160.4 ± 6.23 | 157.3 ± 4.68 | 145.8 ± 7.15 | 148.4 ± 3.52 |
| Total Cholesterol (mg/dL) | 161.3 ± 5.24 | 166.5 ± 3.57 | 153.2 ± 4.76 | 147 ± 5.28 |
| HDL-cholesterol (mg/dL) | 48.2 ± 2.64 | 49.5 ± 3.74 | 54.9 ± 4.98 | 61.2 ± 5.20 |
| LDL-cholesterol (mg/dL) | 83.98 ± 4.62 | 83.76 ± 3.65 | 65.76 ± 2.98 | 54.62 ± 7.45 |
| Triglycerides (mg/dL) | 145.6 ± 3.87 | 166.2 ± 2.56 | 162.7 ± 5.61 | 155.9 ± 4.95 |
| Urea (mmol/L) | 27.4 ± 2.31 | 31.3 ± 2.54 | 25.1 ± 1.69 | 27.6 ± 2.57 |
| Creatinine (mg/dL) | 0.34 ± 0.06 | 0.29 ± 0.02 | 0.3 ± 0.05 | 0.25 ± 0.03 |

G1 = Normal control (only water), G2 = 1875 mg/kg bw, G3 = 3750 mg/kg bw, G4 = 7500 mg/kg bw.

TABLE 22
Effect of Noni leaves extract on biochemical analysis in the acute toxicity study.

| | G1 | G2 | G3 | G4 |
|---|---|---|---|---|
| Parameters | Mean ± SD | Mean ± SD | Mean ± SD | Mean ± SD |
| ALT (U/mL) | 31.2 ± 2.32 | 35.2 ± 3.56 | 34.6 ± 2.34 | 33.2 ± 1.89 |
| AST (U/mL) | 51.4 ± 4.39 | 46.5 ± 2.98 | 48.3 ± 2.64 | 55.4 ± 2.31 |
| ALP (U/mL) | 130.2 ± 3.65 | 123.3 ± 4.26 | 135.2 ± 6.45 | 137.1 ± 3.68 |
| Total Cholesterol (mg/dL) | 156 ± 2.69 | 161.2 ± 5.69 | 168 ± 4.53 | 145.1 ± 3.78 |
| HDL-cholesterol (mg/dL) | 48.2 ± 2.06 | 40.8 ± 2.69 | 43.4 ± 2.94 | 45.6 ± 3.64 |
| LDL-cholesterol (mg/dL) | 69.96 ± 3.25 | 65.9 ± 3.65 | 69.52 ± 2.79 | 65.76 ± 3.54 |
| Triglycerides (mg/dL) | 189.2 ± 7.21 | 172.5 ± 5.34 | 180.4 ± 6.24 | 168.7 ± 3.68 |
| Urea (mmol/L) | 24.5 ± 0.90 | 19.8 ± 1.03 | 28.1 ± 2.43 | 34.3 ± 2.68 |
| Creatinine (mg/dL) | 0.38 ± 0.06 | 0.24 ± 0.04 | 0.29 ± 0.08 | 0.36 ± 0.07 |

G1 = Normal control (only water), G2 = 1250 mg/kg bw, G3 = 2500 mg/kg bw, G4 = 5000 mg/kg bw.

TABLE 23
Effect of Noni seeds extract on biochemical analysis in the acute toxicity study.

| | G1 | G2 | G3 | G4 |
|---|---|---|---|---|
| Parameters | Mean ± SD | Mean ± SD | Mean ± SD | Mean ± SD |
| ALT (U/mL) | 29.5 ± 1.86 | 34.3 ± 2.37 | 31.7 ± 2.34 | 26.4 ± 1.98 |
| AST (U/mL) | 22.5 ± 2.51 | 25.4 ± 1.64 | 21.6 ± 2.39 | 18.7 ± 1.32 |
| ALP (U/mL) | 112.3 ± 2.36 | 118.1 ± 3.51 | 124.5 ± 2.69 | 129.8 ± 2.54 |
| Total Cholesterol (mg/dL) | 157.6 ± 3.58 | 142.2 ± 3.69 | 151.3 ± 5.61 | 164.7 ± 6.34 |
| HDL-cholesterol (mg/dL) | 44.5 ± 2.39 | 51.4 ± 2.61 | 46.6 ± 2.57 | 52 ± 3.51 |
| LDL-cholesterol (mg/dL) | 83.54 ± 3.68 | 63.1 ± 2.31 | 72.82 ± 4.62 | 82.58 ± 5.37 |
| Triglycerides (mg/dL) | 147.8 ± 6.29 | 138.5 ± 5.34 | 159.4 ± 5.71 | 150.6 ± 3.96 |
| Urea (mmol/L) | 30.4 ± 1.72 | 25.8 ± 0.92 | 23.4 ± 0.67 | 28.3 ± 1.37 |
| Creatinine (mg/dL) | 0.5 ± 0.06 | 0.54 ± 0.08 | 0.48 ± 0.09 | 0.42 ± 0.06 |

G1 = Normal control (only water), G2 = 5 mL/kg bw, G3 = 10 mL/kg bw, G4 = 20 mL/kg bw.

TABLE 24
Effect of Noni fruit extracts on biochemical analysis in the sub-acute toxicity study.

| | G1 | G2 | G3 | G4 |
|---|---|---|---|---|
| Parameters | Mean ± SD | Mean ± SD | Mean ± SD | Mean ± SD |
| ALT (U/mL) | 45.3 ± 2.45 | 48.4 ± 1.96 | 41.1 ± 1.05 | 46 ± 2.39 |
| AST (U/mL) | 35.8 ± 2.07 | 33.7 ± 2.54 | 37.4 ± 2.39 | 31.7 ± 2.92 |
| ALP (U/mL) | 99.4 ± 4.62 | 95.8 ± 5.63 | 94.6 ± 4.35 | 102.7 ± 5.09 |
| Total Cholesterol (mg/dL) | 154.2 ± 5.32 | 142.3 ± 3.56 | 148.8 ± 3.28 | 138.4 ± 3.64 |
| HDL-cholesterol (mg/dL) | 46.4 ± 1.68 | 51.2 ± 2.09 | 54.6 ± 2.39 | 60.7 ± 3.51 |
| LDL-cholesterol (mg/dL) | 75.96 ± 3.06 | 61.42 ± 2.05 | 65.28 ± 3.18 | 67.36 ± 3.61 |
| Triglycerides (mg/dL) | 159.2 ± 5.36 | 148.4 ± 3.54 | 144.6 ± 5.62 | 151.7 ± 4.95 |
| Urea (mmol/L) | 29.6 ± 1.36 | 31.2 ± 2.23 | 26.4 ± 1.64 | 23.6 ± 0.96 |
| Creatinine (mg/dL) | 0.34 ± 0.03 | 0.37 ± 0.09 | 0.28 ± 0.05 | 0.26 ± 0.04 |

G1 = Normal control (only water), G2 = 938 mg/kg bw, G3 = 1875 mg/kg bw, G4 = 3750 mg/kg bw.

TABLE 25
Effect of Noni leaves extract on biochemical analysis in the sub-acute toxicity study.

| | G1 | G2 | G3 | G4 |
|---|---|---|---|---|
| Parameters | Mean ± SD | Mean ± SD | Mean ± SD | Mean ± SD |
| ALT (U/mL) | 36.3 ± 1.96 | 41.4 ± 2.47 | 38.6 ± 3.61 | 34.2 ± 1.86 |
| AST (U/mL) | 24.4 ± 1.25 | 27.1 ± 1.91 | 21.9 ± 2.03 | 19.7 ± 1.69 |
| ALP (U/mL) | 110.2 ± 5.23 | 114.5 ± 4.95 | 113.6 ± 6.81 | 105.8 ± 7.26 |
| Total Cholesterol (mg/dL) | 172.7 ± 9.84 | 154.6 ± 6.25 | 157.8 ± 7.38 | 152.7 ± 4.97 |
| HDL-cholesterol (mg/dL) | 44.6 ± 1.59 | 47.5 ± 2.84 | 51.2 ± 3.06 | 60.7 ± 3.71 |
| LDL-cholesterol (mg/dL) | 90.26 ± 2.56 | 78.8 ± 4.81 | 75.92 ± 6.28 | 64.68 ± 4.82 |
| Triglycerides (mg/dL) | 189.2 ± 9.32 | 141.5 ± 6.84 | 153.4 ± 7.69 | 146.6 ± 6.71 |
| Urea (mmol/L) | 27.3 ± 1.29 | 24.5 ± 1.03 | 29.2 ± 1.98 | 21.8 ± 1.27 |
| Creatinine (mg/dL) | 0.35 ± 0.03 | 0.38 ± 0.06 | 0.29 ± 0.04 | 0.28 ± 0.02 |

G1 = Normal control (only water), G2 = 625 mg/kg bw, G3 = 1250 mg/kg bw, G4 = 2500 mg/kg bw.

TABLE 26
Effect of Noni seeds extract on biochemical analysis in the sub-acute toxicity study.

| | G1 | G2 | G3 | G4 |
|---|---|---|---|---|
| Parameters | Mean ± SD | Mean ± SD | Mean ± SD | Mean ± SD |
| ALT (U/mL) | 39.2 ± 1.38 | 35.5 ± 1.69 | 41.8 ± 2.16 | 45.6 ± 3.45 |
| AST (U/mL) | 29.3 ± 2.05 | 32.4 ± 2.74 | 32.7 ± 1.72 | 35.6 ± 3.74 |
| ALP (U/mL) | 170.4 ± 4.45 | 158.5 ± 6.25 | 181.6 ± 7.29 | 155.4 ± 5.21 |
| Total Cholesterol (mg/dL) | 166.2 ± 3.64 | 173.2 ± 4.65 | 189.6 ± 3.61 | 178.4 ± 4.36 |
| HDL-cholesterol (mg/dL) | 41.3 ± 1.29 | 45.2 ± 3.42 | 52.8 ± 5.23 | 55.7 ± 4.97 |
| LDL-cholesterol (mg/dL) | 85.26 ± 3.62 | 94.5 ± 4.39 | 104.28 ± 5.2 | 92.4 ± 4.26 |
| Triglycerides (mg/dL) | 198.2 ± 4.69 | 167.5 ± 4.85 | 162.6 ± 6.29 | 151.5 ± 5.21 |
| Urea (mmol/L) | 18.9 ± 1.26 | 32.8 ± 3.56 | 26.4 ± 2.37 | 25.7 ± 1.51 |
| Creatinine (mg/dL) | 0.36 ± 0.05 | 0.31 ± 0.08 | 0.28 ± 0.06 | 0.40 ± 0.03 |

G1 = Normal control (only water), G2 = 2.5 mL/kg bw, G3 = 5 mL/kg bw, G4 = 10 mL/kg bw.

### 2.6 Anti-Diabetic Effects

Based on the toxicity findings, we calculated the safe dose for noni fruits and leaves and induced in diabetic and normal healthy control mice. We evaluated fasting blood glucose from tail of mice weekly. Moreover, biochemical parameters (total cholesterol, HDL-cholesterol, LDL-cholesterol, triglycerides, ALT, AST and ALP, urea and creatinine) were evaluated from blood samples and histological analysis of liver, heart, kidney and pancreas were observed to know the morphological findings. We found a potential effect of noni leaves and fruits on diabetes.

### Fasting Blood Glucose Level

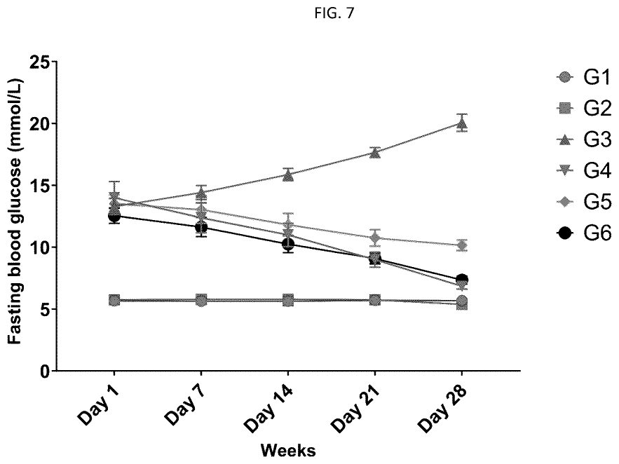

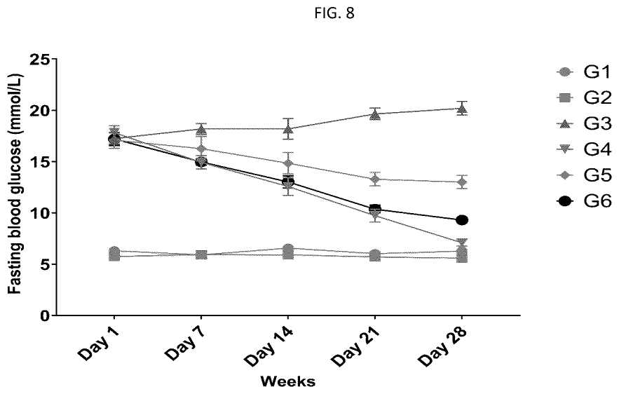

In fasting blood glucose, 41.46% was effective in lowering fasting blood glucose level at the dosage of 400 mg/kg bw fruit extract, whereas 24.88% was effective at 200 mg/kg bw fruit extract (FIG. 7, Table 27). The high dose of 400 mg/kg fruit extract lowered fasting blood glucose to 7.34 mmol/L, which matched the standard drug's effectiveness of 6.84 mmol/L after 28 days of treatment. On the other hand, leaves extract showed a lowering of fasting blood glucose level to 9.3 mmol/L at a dosage of 400 mg/kg bw (FIG. 8, Table 28). The lowest concentration (200 mg/kg bw) of both treatments (fruit and leaves) resulted in minimal effects with the leaves extract reaching 13.0 mmol/L as the least effective outcome. Both extracts showed lowering fasting blood sugar effects when used at higher concentrations.

TABLE 27
Effect Noni fruits extract on fasting blood glucose.

| | G1 | G2 | G3 | G4 | G5 | G6 |
|---|---|---|---|---|---|---|
| | (mmol/L) | (mmol/L) | (mmol/L) | (mmol/L) | (mmol/L) | (mmol/L) |
| Days | Mean ± SEM | Mean ± SEM | Mean ± SEM | Mean ± SEM | Mean ± SEM | Mean ± SEM |
| Day 1 | 5.66 ± 0.18 | 5.74 ± 0.16 | 13.28 ± 0.65 | 13.98 ± 1.32 | 13.5 ± 0.71 | 12.54 ± 0.62 |
| Day 7 | 5.62 ± 0.22 | 5.78 ± 0.12 | 14.4 ± 0.57 | 12.36 ± 1.20 | 13.02 ± 1.10 | 11.62 ± 0.78 |
| Day 14 | 5.62 ± 0.34 | 5.78 ± 0.16 | 15.86 ± 0.50 | 10.98 ± 094 | 11.8 ± 0.91 | 10.24 ± 0.69 |
| Day 21 | 5.7 ± 0.11 | 5.74 ± 0.08 | 17.64 ± 0.69 | 8.98 ± 0.61 | 10.74 ± 0.68 | 9.08 ± 0.43 |
| Day 28 | 5.68 ± 0.15 | 5.38 ± 0.14 | 20.04 ± 1.03 | 6.84 ± 0.24 | 10.14 ± 0.43 | 7.34 ± 0.32 |

G1—Normal mice, G2—Noni fruit treated (200 mg/kg bw) on normal mice: G3—Diabetic control group (STZ treated); G4—Drug control (Glibenclamide 5 mg/kg bw); G5—Noni fruit treated (200 mg/kg bw) on diabetic mice; G6—Noni fruit treated (400 mg/kg bw) on diabetic mice.

TABLE 28
Effect of Noni leaves extracts on fasting blood glucose.

| | G1 | G2 | G3 | G4 | G5 | G6 |
|---|---|---|---|---|---|---|
| | (mmol/L) | (mmol/L) | (mmol/L) | (mmol/L) | (mmol/L) | (mmol/L) |
| Days | Mean ± SEM | Mean ± SEM | Mean ± SEM | Mean ± SEM | Mean ± SEM | Mean ± SEM |
| Day 1 | 6.3 ± 0.08 | 5.74 ± 0.14 | 17.24 ± 0.55 | 17.82 ± 0.68 | 17.08 ± 0.78 | 17.2 ± 0.65 |
| Day 7 | 5.9 ± 0.34 | 5.92 ± 0.14 | 18.18 ± 0.53 | 14.92 ± 0.65 | 16.26 ± 1.19 | 14.96 ± 0.31 |
| Day 14 | 6.56 ± 0.13 | 5.9 ± 0.16 | 18.18 ± 1.01 | 12.56 ± 0.86 | 14.84 ± 1.06 | 13.02 ± 0.59 |
| Day 21 | 6.02 ± 0.21 | 5.7 ± 0.11 | 19.64 ± 0.57 | 9.72 ± 0.64 | 13.28 ± 0.67 | 10.36 ± 0.38 |
| Day 28 | 6.26 ± 0.11 | 5.58 ± 0.17 | 20.2 ± 0.67 | 7.08 ± 0.33 | 13.0 ± 0.64 | 9.3 ± 0.25 |

G1—Normal mice, G2—Noni leaves treated (200 mg/kg bw) on normal mice, G3—Diabetic control group (STZ treated), G4—Drug control (Glibenclamide 5 mg/kg bw), G5—Noni leaves treated (200 mg/kg bw) on diabetic mice, G6—Noni leaves treated (400 mg/kg bw) on diabetic mice.

### Lipid Profile

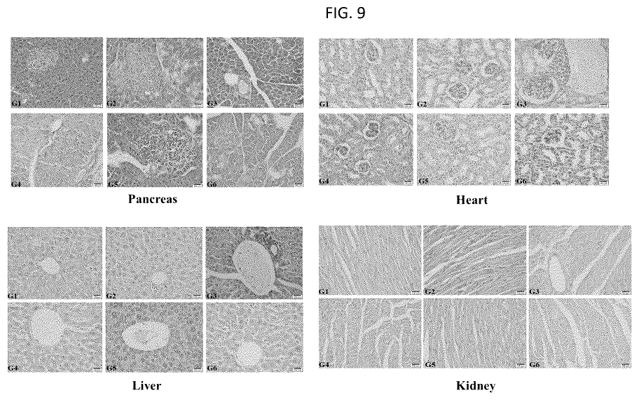

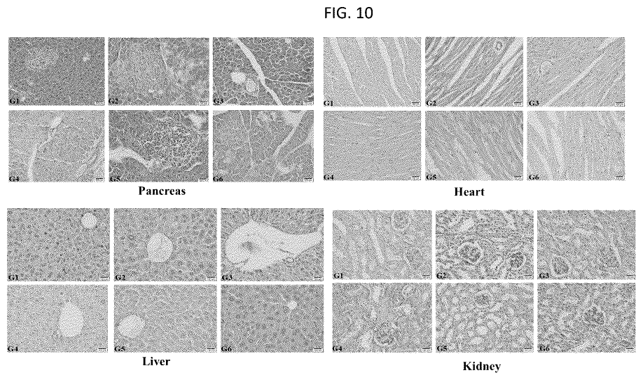

Hyperglycemia resulted in a significant rise in blood triglycerides and total cholesterol. This hyperlipidemia linked with diabetes may be due to a lack of insulin. Normally, insulin stimulates lipoprotein lipase, which hydrolyzes triglycerides. An insulin insufficiency causes the enzymes to be inactive, resulting in hypertriglyceridemia. Blood glucose normalization led to substantial decreases in serum cholesterol, triglycerides, and low-density lipoprotein. In the current research, the G3 diabetic group showed increased total serum cholesterol, triglycerides, while reduced serum high-density lipoproteins. After both fruit and leaves extract therapies in G5 and G6 groups resulted in serum lipid normalization, which may contribute to the favorable impact on pancreatic cells (Table 29, Table 30). In both extracts, the higher dose 400 mg/kg bw reduced the total cholesterol, LDL-C, and triglycerides and increased the high-density lipoprotein cholesterol compared to the lower dose (200 mg/kg bw). Similar findings were also obtained in the morphology of the pancreas, where beta cell regeneration was noticed at higher doses during morphological observation (FIG. 9, FIG. 10).

TABLE 29
Effect of Noni fruit extracts on the lipid profiles.

| | G1 | G2 | G3 | G4 | G5 | G6 |
|---|---|---|---|---|---|---|
| | Mean ± SD | Mean ± SD | Mean ± SD | Mean ± SD | Mean ± SD | Mean ± SD |
| TC | 155.3 ± 8.11 | 141.27 ± 9.87 | 190.67 ± 7.99 | 141.66 ± 3.46 | 141.33 ± 6.33 | 108.53 ± 8.03 |
| (mg/dL) | | | | | | |
| HDL-C | 48.5 ± 2.06 | 51.2 ± 5.35 | 35.97 ± 1.85 | 53.16 ± 3.75 | 47.93 ± 2.74 | 48.87 ± 1.65 |
| (mg/dL) | | | | | | |
| LDL-C | 79.63 ± 7.10 | 63.98 ± 13.40 | 117.34 ± 8.63 | 65.04 ± 4.13 | 69.69 ± 8.55 | 34.07 ± 3.78 |
| (mg/dL) | | | | | | |
| TG | 135.83 ± 10.96 | 130.43 ± 9.46 | 186.8 ± 9.56 | 117.3 ± 4.16 | 118.57 ± 4.99 | 128 ± 6.44 |
| (mg/dL) | | | | | | |

G1—Normal mice, G2—Noni fruit treated (200 mg/kg bw) on normal mice, G3—Diabetic control group (STZ treated), G4—Drug control (Glibenclamide 5 mg/kg bw), G5—Noni fruit treated (200 mg/kg bw) on diabetic mice, G6—Noni fruit treated (400 mg/kg bw) on diabetic mice. TC—Total Cholesterol, HDL-C—High density lipoprotein cholesterol, LDL-C—Low density lipoprotein cholesterol, TG—Triglycerides.

TABLE 30
Effect of Noni leaves extracts on the lipid profiles.

| | G1 | G2 | G3 | G4 | G5 | G6 |
|---|---|---|---|---|---|---|
| | Mean ± SD | Mean ± SD | Mean ± SD | Mean ± SD | Mean ± SD | Mean ± SD |
| TC | 130.63 ± 4.97 | 122.47 ± 7.06 | 161.63 ± 8.59 | 129.37 ± 6.87 | 135.07 ± 12.64 | 120.03 ± 7.86 |
| (mg/dL) | | | | | | |
| HDL-C | 38.83 ± 2.0 | 41.73 ± 1.74 | 36.47 ± 1.66 | 46 ± 4.82 | 36.93 ± 3.65 | 40.57 ± 3.90 |
| (mg/dL) | | | | | | |
| LDL-C | 68.92 ± 6.09 | 55.35 ± 9.45 | 92.22 ± 11.23 | 54.38 ± 6.24 | 69.93 ± 13.85 | 43.87 ± 7.03 |
| (mg/dL) | | | | | | |
| TG | 114.4 ± 5.75 | 126.9 ± 10.13 | 164.73 ± 10.52 | 144.93 ± 9.77 | 141 ± 8.15 | 128 ± 9.21 |
| (mg/dL) | | | | | | |

G1—Normal mice, G2—Noni leaves treated (200 mg/kg bw) on normal mice, G3—Diabetic control group (STZ treated), G4—Drug control (Glibenclamide 5 mg/kg bw), G5—Noni leaves treated (200 mg/kg bw) on diabetic mice, G6—Noni leaves treated (400 mg/kg bw) on diabetic mice. TC—Total Cholesterol, HDL-C—High density lipoprotein cholesterol, LDL-C—Low density lipoprotein cholesterol, TG—Triglycerides.

The enzymes alkaline phosphatase (ALP), alanine aminotransferase (ALT) and aspartate aminotransferase (AST) levels increase in diabetic patients' blood through the release of enzymes from the liver because of oxidative stress and advanced glycosylation end products. Research indicated that the streptozotocin-induced group (G3) elevated ALT, ALP and AST levels in blood. After inducing the extracts for 28 days, the blood enzyme levels AST, ALP, and ALT were decreased at higher dose (G6) compared to G5, resulting in protective effects on the liver and improved liver function at 400 mg/kg bw (Table 31, Table 32). Similar findings were also noticed in the morphological findings of the liver at group 6 during microscopic observation (FIG. 9, FIG. 10).

### Liver Function

TABLE 31
Effect of Noni fruit extracts on the liver functions.

| | G1 | G2 | G3 | G4 | G5 | G6 |
|---|---|---|---|---|---|---|
| | Mean ± SD | Mean ± SD | Mean ± SD | Mean ± SD | Mean ± SD | Mean ± SD |
| ALP | 128.93 ± 7.37 | 116.9 ± 5.32 | 185.57 ± 7.30 | 119.8 ± 9.02 | 125.73 ± 4.42 | 104.9 ± 4.30 |
| (U/mL) | | | | | | |
| ALT | 40.17 ± 5.58 | 38.1 ± 4.42 | 103.8 ± 5.94 | 46.53 ± 3.92 | 33.87 ± 3.22 | 36.33 ± 2.55 |
| (U/mL) | | | | | | |
| AST | 49.97 ± 3.41 | 48.23 ± 3.57 | 106.07 ± 6.11 | 49.33 ± 2.59 | 50.77 ± 6.34 | 43.87 ± 4.46 |
| (U/mL) | | | | | | |

G1—Normal mice, G2—Noni fruit treated (200 mg/kg bw) on normal mice, G3—Diabetic control group (STZ treated), G4—Drug control (Glibenclamide 5 mg/kg bw), G5—Noni fruit treated (200 mg/kg bw) on diabetic mice, G6—Noni fruit treated (400 mg/kg bw) on diabetic mice. ALP—Alkaline phosphatase, ALT—Alanine aminotransferase, AST—Aspartate aminotransferase.

TABLE 32
Effect of Noni leaves extracts on the liver functions.

| Test | G1 | G2 | G3 | G4 | G5 | G6 |
|---|---|---|---|---|---|---|
| Name | Mean ± SD | Mean ± SD | Mean ± SD | Mean ± SD | Mean ± SD | Mean ± SD |
| ALP | 120.33 ± 7.02 | 110.13 ± 6.42 | 173.23 ± 10.77 | 147.6 ± 4.86 | 125.53 ± 6.57 | 115.4 ± 10.74 |
| (U/mL) | | | | | | |
| ALT | 24.57 ± 3.35 | 28.4 ± 5.17 | 75.67 ± 3.59 | 47.37 ± 6.25 | 34.23 ± 5.06 | 24.73 ± 3.03 |
| (U/mL) | | | | | | |
| AST | 33.4 ± 2.14 | 39.63 ± 2.36 | 119.2 ± 12.89 | 40.7 ± 3.61 | 48.83 ± 5.64 | 29.63 ± 3.13 |
| (U/mL) | | | | | | |

G1—Normal mice, G2—Noni leaves treated (200 mg/kg bw) on normal mice, G3—Diabetic control group (STZ treated), G4—Drug control (Glibenclamide 5 mg/kg bw), G5—Noni leaves treated (200 mg/kg bw) on diabetic mice, G6—Noni leaves treated (400 mg/kg bw) on diabetic mice. ALP—Alkaline phosphatase, ALT—Alanine aminotransferase, AST—Aspartate aminotransferase.

### Renal Function

Hyperglycemia-induced elevation of the serum urea and creatinine is considered a significant marker of renal dysfunction. In our study, serum creatinine and urea were not markedly changed except G3 diabetic group. Elevated serum urea and creatinine were slightly reduced by the treatment with both extracts, suggesting a renal protective effect (Table 33, Table 34). In the histological findings of the kidney, we did not notice any major changes in all the groups except the diabetic group G3 (FIG. 9, FIG. 10).

TABLE 33
Effect of Noni fruit extracts on the kidney functions.

| | G1 | G2 | G3 | G4 | G5 | G6 |
|---|---|---|---|---|---|---|
| | Mean ± SD | Mean ± SD | Mean ± SD | Mean ± SD | Mean ± SD | Mean ± SD |
| Creatinine | 0.53 ± 0.04 | 0.48 ± 0.06 | 0.60 ± 0.97 | 0.44 ± 0.04 | 0.49 ± 0.07 | 0.49 ± 0.02 |
| (mg/dL) | | | | | | |
| Urea | 38.67 ± 4.70 | 39.67 ± 5.04 | 67.67 ± 2.91 | 48 ± 3.79 | 39 ± 1.73 | 38.67 ± 8.99 |
| (mmol/L) | | | | | | |

G1—Normal mice, G2—Noni fruit treated (200 mg/kg bw) on normal mice, G3—Diabetic control group (STZ treated), G4—Drug control (Glibenclamide 5 mg/kg bw), G5—Noni fruit treated (200 mg/kg bw) on diabetic mice, G6—Noni fruit treated (400 mg/kg bw) on diabetic mice.

TABLE 34
Effect Noni leaves extracts on the kidney functions.

| | G1 | G2 | G3 | G4 | G5 | G6 |
|---|---|---|---|---|---|---|
| | Mean ± SD | Mean ± SD | Mean ± SD | Mean ± SD | Mean ± SD | Mean ± SD |
| Creatinine | 0.44 ± 0.08 | 0.4 ± 0.04 | 0.73 ± 0.10 | 0.59 ± 0.09 | 0.51 ± 0.06 | 0.46 ± 0.07 |
| (mg/dL) | | | | | | |
| Urea (mmol/L) | 28.33 ± 3.06 | 33.33 ± 4.16 | 55 ± 6.56 | 40 ± 3 | 34.67 ± 4.04 | 32.33 ± 7.57 |

G1—Normal mice, G2—Noni leaves treated (200 mg/kg bw) on normal mice, G3—Diabetic control group (STZ treated), G4—Drug control (Glibenclamide 5 mg/kg bw), G5—Noni leaves treated (200 mg/kg bw) on diabetic mice, G6—Noni leaves treated (400 mg/kg bw) on diabetic mice.

In all these parameters, abnormalities in blood parameters and morphological findings were not obtained in group G2, indicating that both extracts are safe for the healthy groups.

In our comparative findings, noni fruits were more effective in diabetic studies in mouse models in decreasing the fasting blood glucose level. Similar findings were also noticed in B-cell regeneration during the microscopic investigation of pancreatin cells compared to the diabetic groups. In the noni fruit sample, total cholesterol, LDL cholesterol, and triglycerides were reduced, as well as increased HDL cholesterol, known as good cholesterol.

### Example B: Anti-Diabetic Water Formulation from Noni

The noni supercritical fluid extract prepared using any scCO₂ extraction conditions (preferred the optimized conditions) in Example A can be dissolved, suspended or diluted in water and given to a diabetic patient (human). The dose can be calculated using the following well-established equation or any scientifically accepted equation for dose conversion:

$$Human Equivalent Dose ( HED ) = Mouse Dose ( mg / kg ) / Conversion Factor$$

$$HED = ( 400 mg / kg ) / 12.3$$

$$HED \approx 32.52 mg / kg$$

The dose can be further optimized based on the therapeutic response. The scCO₂ extracts can be from one part of the plant or from different parts. It is possible to make the extraction from individual plant parts (fruits, leaves, seeds). It is also possible to make the extraction from mixed plant parts considering that the teaching of the current invention can be applied without undue experimentation.

### Example C: Anti-Diabetic Liquid Formulation from Noni

The noni supercritical fluid extract as explained in Example A and Example B, can be dissolved, suspended or diluted in water with additional pharmaceutically accepted ingredients or food accepted ingredients and given to diabetic patient (human). The dose can be further optimized based on the therapeutic response.

### Example D: Anti-Diabetic Solid Formulation from Noni

The noni supercritical fluid extract as explained in Example A and Example B, can be prepared as a solid dosage form (like tablet or capsule) with or without additional pharmaceutically accepted ingredients or food accepted ingredients and given to diabetic patient (human). The dose can be further optimized based on the therapeutic response.

### Example E: Anti-Diabetic Other Formulation from Noni

The noni supercritical fluid extract as explained in Example A and Example B, can be prepared in different pharmaceutical or nutraceutical dosage forms with or without additional pharmaceutically accepted ingredients or food accepted ingredients and given to diabetic patient (human). The dose can be further optimized based on the therapeutic response.

Pharmaceutical and nutraceutical dosage forms include but not limited to:
Tablets (e.g., uncoated, coated, effervescent, soluble, chewable, prolonged-release, orally disintegrating)
Capsules (e.g., hard, soft, prolonged-release)
Liquids (e.g., solutions, suspensions, emulsions, syrups, elixirs, tinctures, gargles, mouthwashes, nasal drops, eye drops, ear drops)
Semisolids (e.g., creams, ointments, gels, pastes, suppositories, pessaries)
Injections (e.g., solutions, suspensions, emulsions, powders for reconstitution)
Inhalations (e.g., aerosols, dry powder inhalers, nebulizer solutions)
Transdermal patches
Implants
Powders (e.g., for reconstitution, dusting powders)
Granules
Sprays (e.g., nasal sprays, oral sprays, topical sprays)
Films (e.g., oral soluble films)
Lozenges/Pastilles
Medicated chewing gums
Foams
Medicated plasters

Pharmaceutically accepted ingredients include but not limited to chelating agent, preservative, adsorbents, acidifying agent, alkalizing agent, antifoaming agent, buffering agent, colorant, electrolyte, flavorant, polishing agent, salt, stabilizer, sweetening agent, tonicity modifier, antiadherent, binder, diluent, direct compression excipient, disintegrant, glidant, lubricant, opaquant, polishing agent, plasticizer, other pharmaceutical excipient, or a combination thereof.

### Example F: Anti-Diabetic Synergistic Formulation from Noni

The noni supercritical fluid extract as explained in Example A and Example B, can be prepared in different pharmaceutical or nutraceutical dosage forms with or without additional pharmaceutically accepted ingredients or food accepted ingredients and given to diabetic patient (human) together with at least one anti-diabetic drug or supplement or any anti-diabetic composition in order to have a synergistic effect. The dose can be further optimized based on the therapeutic response. The dose can be further reduced below the calculated dose considering there is another anti-diabetic effect from the other composition. Noni extract and the other anti-diabetic composition can be mixed in one dosage form or given separately at the same time or at different times.

1. An anti-diabetic composition comprising an extract of Morinda citrifolia prepared using a method of supercritical fluid extraction, said method comprising:
adding a plant material from Morinda citrifolia to an extraction vessel;
adding ethanol to the plant material;
treating the plant material with carbon dioxide supercritical fluid for a period of time sufficient to extract anti-diabetic compounds;
separating the plant material from the carbon dioxide supercritical fluid; and
removing the carbon dioxide supercritical fluid and ethanol thereby forming the anti-diabetic composition in a form of Morinda citrifolia supercritical fluid extract;
and wherein:
inhibition activity of the extract of Morinda citrifolia is characterized by IC₅₀ lower than 50 μg/mL measured in vitro using isolated α-glucosidase enzyme.

2. The anti-diabetic composition according to claim 1, wherein the extract of Morinda citrifolia is prepared from Morinda citrifolia fruits, leaves, seeds, or a combination of them.

3. The anti-diabetic composition according to claim 1, wherein the extract of Morinda citrifolia comprises at least one anti-diabetic compound selected from a group consisting of Monotropein, m-Coumaric acid, Betulinic Acid, Robinetin 3-Rutinoside, Luteolin 5,3′-Dimethyl Ether, C16 Sphinganine, 1-Linoleoyl Glycerol, 9Z,12Z,15E-Octadecatrienoic Acid, Lamiide, and Reboxetine.

4. The anti-diabetic composition according to claim 1, wherein the extract of Morinda citrifolia has a blood sugar lowering effect in diabetic mammals when given orally at an effective blood sugar lowering dose.

5. The anti-diabetic composition according to claim 1, wherein the extract of Morinda citrifolia has no acute toxicity when used at a dose between an effective blood sugar lowering dose in diabetic mammals and 25 times the effective blood sugar lowering dose in diabetic mammals.

6. The anti-diabetic composition according to claim 1, wherein the extract of Morinda citrifolia has no sub-acute toxicity when used at a dose between an effective blood sugar lowering dose in diabetic mammals and 10 times the effective blood sugar lowering dose in diabetic mammals.

7. The anti-diabetic composition according to claim 1, wherein the extract of Morinda citrifolia further reduces blood total cholesterol when used at an effective blood sugar lowering dose in diabetic mammals.

8. The anti-diabetic composition according to claim 1, wherein the extract of Morinda citrifolia further reduces blood low-density lipoprotein cholesterol when used at an effective blood sugar lowering dose in diabetic mammals.

9. The anti-diabetic composition according to claim 1, wherein the extract of Morinda citrifolia further reduces blood triglycerides when used at an effective blood sugar lowering dose in diabetic mammals.

10. The anti-diabetic composition according to claim 1, wherein the extract of Morinda citrifolia further increases blood high-density lipoprotein when used at an effective blood sugar lowering dose in diabetic mammals.

11. The anti-diabetic composition according to claim 1, wherein the extract of Morinda citrifolia has reduced or eliminated Morinda citrifolia's unpleasant smell or unpleasant taste.

12. A method of reducing blood glucose in mammals, comprising administering an effective amount of the anti-diabetic composition of claim 1, wherein the anti-diabetic composition is administered orally.
